(19) United States
(12) Patent Application Publication
(10) Pub. No.: US 2020/0385714 A1
(43) Pub. Date: Dec. 10, 2020

(54) OLIGONUCLEOTIDES FOR MODULATING FNDC3B EXPRESSION

(71) Applicant: Roche Innovation Center Copenhagen A/S, Horsholm (DK)

(72) Inventors: Peter HAGEDORN, Hørsholm (DK); Lykke PEDERSEN, Copenhagen (DK)

(73) Assignee: Roche Innovation Center Copenhagen A/S, Horsholm (DK)

(21) Appl. No.: 15/733,226

(22) PCT Filed: Dec. 10, 2018
(86) PCT No.: PCT/EP2018/084075

§ 371 Date: Jun. 11, 2020

(30) Foreign Application Priority Data
Dec. 11, 2017 (EP) 17206545.0

(51) Int. Cl. C12N 15/113; A61K 47/54

(52) U.S. Cl. CPC C12N 15/113; A61K 47/549; C12N 2310/322; C12N 2310/3231; C12N 2310/3341; C12N 2310/351; C12N 2310/11; C12N 2310/341; C12N 2310/315

(57) ABSTRACT

The present invention relates to antisense oligonucleotides that are capable of modulating expression of FNDC3B in a target cell. The oligonucleotides hybridize to FNDC3B pre-mRNA or mRNA. The present invention further relates to conjugates of the oligonucleotide and pharmaceutical compositions and methods for treatment of cancers, such as hepatocellular carcinoma or acute myeloid leukemia using the oligonucleotide.

### FIELD OF INVENTION

The present invention relates to antisense oligonucleotides (oligomers) that are complementary to a mammalian FNDC3B (fibronectin type III domain containing 3B or FAD104) transcript and their use in therapy. Such oligonucleotides may be used for reducing FNDC3B transcript in a cell, leading to modulation of the expression of FNDC3B. Modulation of FNDC3B expression is beneficial for a range of medical disorders, such as cancers, such as hepatocellular carcinoma and/or acute myeloid leukemia.

### BACKGROUND

Cai C et al., Cell Cycle. 2012 May 1; 11(9):1773-81 discloses that FNDC3B amplification and over-expression is an oncogenic driver, and is required for the maintenance of tumorigenic phenotype, in particular in hepatocellular carcinoma. The article refers to shRNA mediated inhibition of FNDC3B.

RNA interference mediated inhibition of FNDC3B is mentioned in Chin-Hui Lin, Oncotarget. 2016 Aug. 2; 7(31): 49498-49508 by short hairpin RNA. No shRNA or siRNA compounds appear to be disclosed.

WO2010110346 discloses a therapeutic agent for AML (acute myeloid leukemia) targeting leukemic stem cells, comprising, as an active ingredient a substance which is capable of inhibiting the expression of a gene selected from leukemic marker genes (such as FNDC3B) or a substance which is capable of inhibiting the activity of the translation product of said gene. No specific siRNA compounds appear to be disclosed.

Antisense oligonucleotides targeting repeated sites in the same RNA have been shown to have enhanced potency for downregulation of target mRNA in some cases of in vitro transfection experiments. This has been the case for GCGR, STST3, MAPT, OGFR, and BOK RNA (Vickers at al. PLOS one, October 2014, Volume 9, Issue 10). WO 2013/120003 also refers to modulation of RNA by repeat targeting.

### Objective of the Invention

FNDC3B is involved in the development and progression of a number of cancers. The present invention provides antisense oligonucleotides capable of modulating FNDC3B mRNA and protein expression, in vivo or in vitro. Accordingly, the present invention can potentially be used in a combination therapy together with the known cancer care and potentially can alleviate symptoms of cancers, in particular, such cancers as acute myeloid leukemia and/or hepatocellular carcinoma.

### SUMMARY OF INVENTION

The present invention provides oligonucleotides, which are complementary to and target mammalian FNDC3B nucleic acids, and uses thereof.

The present invention provides oligonucleotides which comprise a contiguous nucleotide sequence which is complementary to certain regions or sequences present in target mammalian FNDC3B nucleic acids.

The oligonucleotides of the invention are capable of inhibiting mammalian FNDC3B nucleic acids in a cell which is expressing the mammalian FNDC3B nucleic acid.

The present invention provides for an antisense oligonucleotide targeting a mammalian FNDC3B nucleic acid, and in vitro and in vivo uses thereof, and their use in medicine.

The present invention relates to oligonucleotides targeting a FNDC3B nucleic acid and their use to treat or prevent diseases or alleviate symptoms of the disease related to the functioning of the FNDC3B.

Accordingly, in a first aspect the invention provides an antisense oligonucleotide of 10 to 50 nucleotides in length, which comprises a contiguous nucleotide sequence of 10 to 30 nucleotides in length with at least 90% complementarity, such as fully complementary, to a mammalian FNDC3B nucleic acid, wherein the antisense oligonucleotide is capable of reducing the expression of the mammalian FNDC3B encoding target nucleic acid, in a cell.

Accordingly, in a further aspect the invention provides the antisense oligonucleotide wherein the contiguous nucleotide sequence is at least 90% complementary, such as fully complementary, to a sequence selected from the group consisting of SEQ ID NO 1, 2, 3, 4, 5, 6, 7 and 8, or a naturally occurring variant thereof.

Accordingly, in a further apect, the invention provides an antisense oligonucleotide of 10 to 30 nucleotides in length, which comprises a contiguous nucleotide sequence of 10 to 30 nucleotides in length which is fully complementary to a human FNDC3B target nucleic acid, such as SEQ ID NO 1, wherein the antisense oligonucleotide is capable of reducing the expression of human FNDC3B in a cell.

In a further aspect the invention provides the antisense oligonucleotide, wherein the contiguous nucleotide sequence is at least 90% complementary, such as fully complementary, to more than one region of SEQ ID NO 1.

In a further aspect the invention provides the antisense oligonucleotide, wherein the contiguous nucleotide sequence is at least 90% complementary, such as fully complementary, to a region of SEQ ID NO 1, selected from the group consisting of position 54366-54385, 54407-54426, 54448-54467, 54489-54508, 54530-54549, 54571-54590, 54612-54631; 54367-54384, 54408-54425, 54449-54466, 54490-54507, 54531-54548, 54572-54589, 54613-54630; 54368-54383, 54409-54424, 54450-54465, 54491-54506, 54532-54547, 54573-54588, 54614-54629; 54366-54379, 54407-54420, 54448-54461, 54489-54502, 54530-54543, 54571-54584, 54612-54625; 351016-351033; 351016-351029; 351016-351035; 351018-35133 of SEQ ID NO: 1.

In a further aspect the invention provides the antisense oligonucleotide, wherein the antisense oligonucleotide, or contiguous nucleotide sequence thereof is selected from the group consisting of GaatttgctagtggtggtGG (Compound ID 11_1); GCTagtggtggtGG (Compound ID 12_1); AATttgctagtggtgGTG (Compound ID 13_1); ATTTgctagtggtgGT (Compound ID 14_1); TTatttctacagtttccAGA (Compound ID 15_1); ATTTctacagtttccAGA (Compound ID 16_1); CTacagtttcCAGA (Compound ID 17_1); and ATTTctacagtttCCA (Compound ID 18_1); wherein capital letters represent LNA nucleosides, such as beta-D-oxy LNA nucleosides, lower case letters represent DNA nucleosides, optionally all LNA C are 5-methyl cytosine, wherein the compounds comprise phosphorothioate internucleoside linkages.

In a further aspect the invention provides the antisense oligonucleotide, wherein the antisense oligonucleotide, or contiguous nucleotide sequence thereof is selected from the group consisting of GaatttgctagtggtggtGG (Compound ID 11_1); GCTagtggtggtGG (Compound ID 12_1); AATttgctagtggtgGTG (Compound ID 13_1); ATTTgctagtggtgGT (Compound ID 14_1); TTatttctacagtttccAGA (Compound ID 15_1); ATTTctacagtttccAGA (Compound ID 16_1); CTacagtttcCAGA (Compound ID 17_1); and ATTTctacagtttCCA (Compound ID 18_1); wherein capital letters represent beta-D-oxy LNA nucleosides, lower case letters represent DNA nucleosides, all LNA C are 5-methyl cytosine, and all internucleoside linkages are phosphorothioate internucleoside linkages.

In a further aspect the invention provides a conjugate comprising the antisense oligonucleotide according to the invention, and at least one conjugate moiety covalently attached to said oligonucleotide.

In a further aspect the invention provides a pharmaceutical composition comprising the oligonucleotide according to the invention or the conjugate according to the invention, and a pharmaceutically acceptable diluent, solvent, carrier, salt and/or adjuvant.

In a further aspect, the invention provides a pharmaceutically acceptable salt of the antisense oligonucleotide or conjugate according to the invention the invention.

In a further aspect the invention provides a method, such as an in vivo or in vitro method, for inhibiting a mammalian FNDC3B expression in a target cell which is expressing the mammalian FNDC3B, said method comprising administering an oligonucleotide, the conjugate, the pharmaceutically acceptable salt, or the pharmaceutical composition according to the invention in an effective amount to said cell.

In a further aspect the invention provides a method for treating or preventing a disease comprising administering a therapeutically or prophylactically effective amount of an oligonucleotide, the conjugate, the pharmaceutically acceptable salt, or the pharmaceutical composition according to the invention to a subject suffering from or susceptible to the disease.

In a further aspect the invention provides a use of the oligonucleotide, the conjugate, the pharmaceutically acceptable salt, or the pharmaceutical composition for the preparation of a medicament for treatment or prevention of cancer, such as hepatocellular carcinoma or acute myeloid leukemia.

### Definitions

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention is man-made, and is chemically synthesized, and is typically purified or isolated. The oligonucleotide of the invention may comprise one or more modified nucleosides or nucleotides.

Antisense Oligonucleotides

The term “Antisense oligonucleotide” as used herein is defined as oligonucleotides capable of modulating expression of a target gene by hybridizing to a target nucleic acid, in particular to a contiguous sequence on a target nucleic acid, in a cell which is expressing the target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self complementarity is less than 50% across of the full length of the oligonucleotide. The antisense oligonucleotide of the invention is man-made, and is chemically synthesized, and is typically purified or isolated. The antisense oligonucleotide of the invention may comprise one or more modified nucleosides or nucleotides.

Contiguous Nucleotide Sequence

The term “contiguous nucleotide sequence” refers to the region of the oligonucleotide which is complementary to the target nucleic acid. The term is used interchangeably herein with the term “contiguous nucleobase sequence” and the term “oligonucleotide motif sequence”. In some embodiments all the nucleotides of the oligonucleotide constitute the contiguous nucleotide sequence. In some embodiments the oligonucleotide comprises the contiguous nucleotide sequence, such as a F-G-F′ gapmer region, and may optionally comprise further nucleotide(s), for example a nucleotide linker region which may be used to attach a functional group to the contiguous nucleotide sequence. The nucleotide linker region may or may not be complementary to the target nucleic acid.

Nucleotides Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkage

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 75% such as at least 80% or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage for use in the oligonucleotide of the invention is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 75%, such as at least 80% or such as at least 90% of the internucleoside linkages in the oligonucleotide, or contiguous nucleotide sequence thereof, are phosphorothioate. In some embodiments, all of the internucleoside linkages of the oligonucleotide, or contiguous nucleotide sequence thereof, are phosphorothioate. In some embodiments, the oligonucleotide of the invention comprises both phosphorothioate internucleoside linkages and at least one phosphodiester linkage, such as 2, 3 or 4 phosphodiester linkages, in addition to the phosphorodithioate linkage(s). In a gapmer oligonucleotide, phosphodiester linkages, when present, are suitably not located between contiguous DNA nucleosides in the gap region G.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, where all the internucleoside linkages in region G may be phosphorothioate.

Advantageously, all the internucleoside linkages in the contiguous nucleotide sequence of the oligonucleotide, or all the internucleoside linkages of the oligonucleotide, are phosphorothioate linkages.

It is recognized that, as disclosed in EP 2 742 135, antisense oligonucleotides may comprise other internucleoside linkages (other than phosphodiester and phosphorothioate), for example alkyl phosphonate/methyl phosphonate internucleosides, which according to EP 2 742 135 may for example be tolerated in an otherwise DNA phosphorothioate the gap region.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Modified Oligonucleotide

The term modified oligonucleotide describes an oligonucleotide comprising one or more sugar-modified nucleosides and/or modified internucleoside linkages. The term chimeric” oligonucleotide is a term that has been used in the literature to describe oligonucleotides with modified nucleosides.

Complementarity

The term “complementarity” describes the capacity for Watson-Crick base-pairing of nucleosides/nucleotides. Watson-Crick base pairs are guanine (G)-cytosine (C) and adenine (A)-thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

The term “% complementary” as used herein, refers to the proportion of nucleotides (in percent) of a contiguous nucleotide sequence in a nucleic acid molecule (e.g. oligonucleotide) which across the contiguous nucleotide sequence, are complementary to a reference sequence (e.g. a target sequence or sequence motif). The percentage of complementarity is thus calculated by counting the number of aligned nucleobases that are complementary (from Watson Crick base pair) between the two sequences (when aligned with the target sequence 5′-3′ and the oligonucleotide sequence from 3′-5′), dividing that number by the total number of nucleotides in the oligonucleotide and multiplying by 100. In such a comparison a nucleobase/nucleotide which does not align (form a base pair) is termed a mismatch. Insertions and deletions are not allowed in the calculation of % complementarity of a contiguous nucleotide sequence. It will be understood that in determining complementarity, chemical modifications of the nucleobases are disregarded as long as the functional capacity of the nucleobase to form Watson Crick base pairing is retained (e.g. 5′-methyl cytosine is considered identical to a cytosine for the purpose of calculating % identity).

The term “fully complementary”, refers to 100% complementarity.

The following is an example of an oligonucleotide according to the invention that is fully complementary to the target nucleic acid.

The following is an example of an oligonucleotide (SEQ ID NO: 15) that is fully complementary to the target nucleic acid (SEQ ID NO: 6).

(SEQ ID NO: 6)
5′ CAACATGCTCGGCCTGTTACACACACACACACACAC 3′
(SEQ ID NO: 15)
3′ CGAGCCGGACAATGTGTGTG 5′

Identity

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tₘ) defined as the temperature at which half of the oligonucleotides are duplexed with the target nucleic acid. At physiological conditions Tₘ is not strictly proportional to the affinity (Mergny and Lacroix, 2003, Oligonucleotides 13:515-537). The standard state Gibbs free energy ΔG° is a more accurate representation of binding affinity and is related to the dissociation constant (K_{d}) of the reaction by ΔG°=−RTIn(K_{d}), where R is the gas constant and T is the absolute temperature. Therefore, a very low ΔG° of the reaction between an oligonucleotide and the target nucleic acid reflects a strong hybridization between the oligonucleotide and target nucleic acid. ΔG° is the energy associated with a reaction where aqueous concentrations are 1M, the pH is 7, and the temperature is 37° C. The hybridization of oligonucleotides to a target nucleic acid is a spontaneous reaction and for spontaneous reactions ΔG° is less than zero. ΔG° can be measured experimentally, for example, by use of the isothermal titration calorimetry (ITC) method as described in Hansen et al., 1965, Chem. Comm. 36-38 and Holdgate et al., 2005, Drug Discov Today. The skilled person will know that commercial equipment is available for ΔG° measurements. ΔG° can also be estimated numerically by using the nearest neighbor model as described by SantaLucia, 1998, Proc Natl Acad Sci USA. 95: 1460-1465 using appropriately derived thermodynamic parameters described by Sugimoto et al., 1995, Biochemistry 34:11211-11216 and McTigue et al., 2004, Biochemistry 43:5388-5405. In order to have the possibility of modulating its intended nucleic acid target by hybridization, oligonucleotides of the present invention hybridize to a target nucleic acid with estimated ΔG° values below −10 kcal for oligonucleotides that are 10-30 nucleotides in length. In some embodiments the degree or strength of hybridization is measured by the standard state Gibbs free energy ΔG°. The oligonucleotides may hybridize to a target nucleic acid with estimated ΔG° values below the range of −10 kcal, such as below −15 kcal, such as below −20 kcal and such as below −25 kcal for oligonucleotides that are 8-30 nucleotides in length. In some embodiments the oligonucleotides hybridize to a target nucleic acid with an estimated ΔG° value of −10 to −60 kcal, such as −12 to −40, such as from −15 to −30 kcal or −16 to −27 kcal such as −18 to −25 kcal.

Target Nucleic Acid

According to the present invention, the target nucleic acid is a nucleic acid which encodes mammalian FNDC3B and may for example be a gene, a RNA, a mRNA, and pre-mRNA, a mature mRNA or a cDNA sequence. The target may therefore be referred to as an FNDC3B target nucleic acid. The oligonucleotide of the invention may for example target exon regions of a mammalian FNDC3B, or may, for example target any intron region in the FNDC3B pre-mRNA (see, for example, Table 1).

TABLE 1
Human FNDC3B Exons and Introns of one of the splice variants.

| | |
|---|---|
| Exonic regions in the | Intronic regions in the |
| human FNDC3B | human FNDC3B |
| premRNA (SEQ ID | premRNA (SEQ ID |
| NO1) | NO 1) |

| ID | start | end | ID | start | end |
|---|---|---|---|---|---|
| e1 | 1 | 144 | i1 | 145 | 72824 |
| e2 | 72825 | 72963 | i2 | 72964 | 93844 |
| e3 | 93844 | 93919 | i3 | 93920 | 147084 |
| e4 | 147085 | 147671 | | | |

Suitably, the target nucleic acid encodes an FNDC3B protein, in particular mammalian FNDC3B, such as human FNDC3B (See for example tables 2 and 3) which provides the mRNA and pre-mRNA sequences for human, monkey, mice and rat FNDC3B).

In some embodiments, the target nucleic acid is selected from the group consisting of SEQ ID NO: 1, 2, 3, 4, 5, 6, 7 and 8 or naturally occurring variants thereof (e.g. sequences encoding a mammalian FNDC3B protein.

The target nucleic acid may, in some embodiments, be a RNA or DNA, such as messenger RNA, such as a mature mRNA or a pre-mRNA which encodes mammalian FNDC3B protein, such as human FNDC3B, e.g. the human pre-mRNA sequence, such as that disclosed as SEQ ID NO:1 or human mature mRNA as disclosed in SEQ ID NO:2, SEQ ID NO:3, SEQ ID NO:4, SEQ ID NO:5, SEQ ID NO:6 and SEQ ID NO:7 or naturally occurring variants thereof (e.g. sequences encoding a mammalian FNDC3B protein).

If employing the oligonucleotide of the invention in research or diagnostics the target nucleic acid may be a cDNA or a synthetic nucleic acid derived from DNA or RNA.

For in vivo or in vitro application, the oligonucleotide of the invention is typically capable of inhibiting the expression of the FNDC3B target nucleic acid in a cell which is expressing the FNDC3B target nucleic acid. The contiguous sequence of nucleobases of the antisense oligonucleotide of the invention is typically complementary to the FNDC3B target nucleic acid, as measured across the length of the antisense oligonucleotide, optionally with the exception of one or two mismatches, and optionally excluding nucleotide based linker regions which may link the oligonucleotide to an optional functional group such as a conjugate, or other non-complementary terminal nucleotides (e.g. region D′ or D″). The target nucleic acid may, in some embodiments, be a RNA or DNA, such as a messenger RNA, such as a mature mRNA or a pre-mRNA. In some embodiments the target nucleic acid is a RNA or DNA which encodes mammalian FNDC3B protein, such as human FNDC3B, e.g. the human FNDC3B pre-mRNA sequence, such as that disclosed as SEQ ID NO 1. Further information on exemplary target nucleic acids is provided in tables 2 and 3.

TABLE 2
Genome and assembly information for FNDC3B across species.

| | | | Genomic coordinates | | | |
|---|---|---|---|---|---|---|
| Species | Chr. | Strand | Start | End | Assembly | Ensemble ID |
| Human | 3 | fwd | 172039628 | 172401665 | GRCh38.p2 release 107 | ENSG00000075420 |
| Mus | 3 | rev | 27416162 | 27711307 | GRCm38.p5 | ENSMUSG00000039286 |
| musculus | | | | | | |
| Cynomolgus | 2 | fwd | 76257179 | 76622494 | Macaca_{—}fascicularis_5.0 | |
| monkey | | | | | release 100 (GCF_000364345) | |
| Rat | 2 | rev | 113112902 | 113345577 | Rnor_6.0 | ENSRNOG00000024089 |
| norvegics | | | | | release 105 | |

Fwd = forward strand. The genome coordinates provide the pre-mRNA sequence (genomic sequence). The NCBI reference provides the mRNA sequence (cDNA sequence).

TABLE 3
Sequence details for FNDC3B across species.

| | | Length | SEQ ID |
|---|---|---|---|
| Species | RNA type | (nt) | NO |
| Human | Pre- | 362038 | 1 |
| | mRNA | | |
| Human | mRNA | 7904 | 2 |
| Human | mRNA | 408 | 3 |
| Human | mRNA | 678 | 4 |
| Human | mRNA | 3684 | 5 |
| Human | mRNA | 5083 | 6 |
| Human | mRNA | 946 | 7 |
| Cynomonkey | Pre- | 365316 | 8* |
| | mRNA | | |

Note SEQ ID NO 8 comprises regions of multiple NNNNs, where the sequencing has been unable to accurately refine the sequence, and a degenerate sequence is therefore included. For the avoidance of doubt the compounds of the invention are complementary to the actual cynomonkey target sequence and are not therefore degenerate compounds.

Target Sequence

The term “target sequence” as used herein refers to a sequence of nucleotides present in the target nucleic acid which comprises the nucleobase sequence which is complementary to the antisense oligonucleotide of the invention. In some embodiments, the target sequence consists of a region on the target nucleic acid with a nucleobase sequence that is complementary to the contiguous nucleotide sequence of the antisense oligonucleotide of the invention. This region of the target nucleic acid may interchangeably be referred to as the target nucleotide sequence, target sequence or target region. In some embodiments the target sequence is longer than the contiguous complementary sequence of a single oligonucleotide, and may, for example represent a preferred region of the target nucleic acid which may be targeted by several oligonucleotides of the invention.

The antisense oligonucleotide of the invention comprises a contiguous nucleotide sequence which is complementary to the target nucleic acid such as a target sequence described herein.

The target sequence to which the oligonucleotide is complementary generally comprises a contiguous nucleobase sequence of at least 10 nucleotides. The contiguous nucleotide sequence is between 10 to 50 nucleotides, such as 12 to 30, such as 14 to 20, such as 15 to 18 contiguous nucleotides

In one embodiment of the invention the target sequence is SEQ ID NO: 9.

In another embodiment of the invention the target sequence is SEQ ID NO: 10.

In another embodiment of the invention the target sequence is SEQ ID NO: 19.

In another embodiment of the invention the target sequence is SEQ ID NO: 20.

Repeated Target Region

The target region or target sequence can be unique for the target nucleic acid (only present once).

In some aspects of the invention the target region is repeated at least two times over the span of target nucleic acid. Repeated as encompassed by the present invention means that there are at least two identical nucleotide sequences (target regions) of at least 10, such as at least 11, or at least 12, nucleotides in length which occur in the target nucleic acid at different positions. Each repeated target region is separated from the identical region by at least one nucleobase on the contiguous sequence of target nucleic acid and is positioned at different and non-overlapping positions within the target nucleic acid.

Target Cell

The term “target cell” as used herein refers to a cell which is expressing the target nucleic acid. In some embodiments the target cell may be in vivo or in vitro. In some embodiments the target cell is a mammalian cell such as a rodent cell, such as a mouse cell or a rat cell, or a primate cell such as a monkey cell or a human cell.

In preferred embodiments the target cell expresses FNDC3B mRNA, such as the FNDC3B pre-mRNA or FNDC3B mature mRNA. The poly A tail of FNDC3B mRNA is disregarded for antisense oligonucleotide targeting.

Naturally Occurring Variant

The term “naturally occurring variant” refers to variants of FNDC3B gene or transcripts which originate from the same genetic loci as the target nucleic acid and is a directional transcript from the same chromosomal position and direction as the target nucleic acid, but may differ for example, by virtue of degeneracy of the genetic code causing a multiplicity of codons encoding the same amino acid, or due to alternative splicing of pre-mRNA, or the presence of polymorphisms, such as single nucleotide polymorphisms, and allelic variants. Based on the presence of the sufficient complementary sequence to the oligonucleotide, the oligonucleotide of the invention may therefore target the target nucleic acid and naturally occurring variants thereof.

In some embodiments, the naturally occurring variants have at least 95% such as at least 98% or at least 99% homology to a mammalian FNDC3B target nucleic acid, such as a target nucleic acid selected form the group consisting of SEQ ID NO 1, 2, 3, 4, 5, 6, 7 or 8.

Modulation of Expression

The term “modulation of expression” as used herein is to be understood as an overall term for an oligonucleotide's ability to alter the amount of FNDC3B when compared to the amount of FNDC3B before administration of the oligonucleotide. Alternatively modulation of expression may be determined by reference to a control experiment. It is generally understood that the control is a target cell treated with a saline composition or a target cell treated with a non-targeting oligonucleotide (mock).

A modulation according to the present invention shall be understood as an antisense oligonucleotide's ability to inhibit, down-regulate, reduce, suppress, remove, stop, block, prevent, lessen, lower, avoid or terminate expression of FNDC3B, e.g. by degradation of mRNA or blockage of transcription.

High Affinity Modified Nucleosides

A high affinity modified nucleoside is a modified nucleotide which, when incorporated into the oligonucleotide enhances the affinity of the oligonucleotide for its complementary target, for example as measured by the melting temperature (T^{m}). A high affinity modified nucleoside of the present invention preferably result in an increase in melting temperature between +0.5 to +12° C., more preferably between +1.5 to +10° C. and most preferably between +3 to +8° C. per modified nucleoside. Numerous high affinity modified nucleosides are known in the art and include for example, many 2′ substituted nucleosides as well as locked nucleic acids (LNA) (see e.g. Freier & Altmann; Nucl. Acid Res., 1997, 25, 4429-4443 and Uhlmann; Curr. Opinion in Drug Development, 2000, 3(2), 293-213).

Sugar Modifications

The oligomer of the invention may comprise one or more nucleosides which have a modified sugar moiety, i.e. a modification of the sugar moiety when compared to the ribose sugar moiety found in DNA and RNA.

Numerous nucleosides with modification of the ribose sugar moiety have been made, primarily with the aim of improving certain properties of oligonucleotides, such as affinity and/or nuclease resistance.

Such modifications include those where the ribose ring structure is modified, e.g. by replacement with a hexose ring (HNA), or a bicyclic ring, which typically have a biradicle bridge between the C2 and C4 carbons on the ribose ring (LNA), or an unlinked ribose ring which typically lacks a bond between the C2 and C3 carbons (e.g. UNA). Other sugar modified nucleosides include, for example, bicyclohexose nucleic acids (WO2011/017521) or tricyclic nucleic acids (WO2013/154798). Modified nucleosides also include nucleosides where the sugar moiety is replaced with a non-sugar moiety, for example in the case of peptide nucleic acids (PNA), or morpholino nucleic acids.

2′ Modified Nucleosides.

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

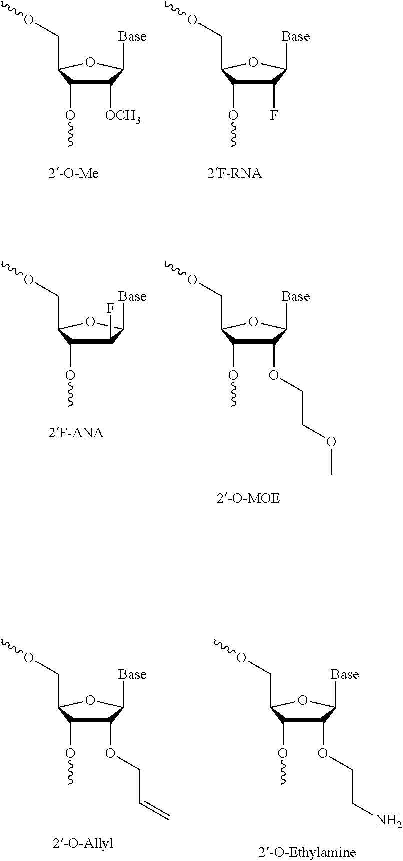

In relation to the present invention 2′ substituted sugar modified nucleosides does not include 2′ bridged nucleosides like LNA.

Locked Nucleic Acid Nucleosides (LNA).

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 12, 73-76, Seth et al. J. Org. Chem. 2010, Vol 75(5) pp. 1569-81 and Mitsuoka et al., Nucleic Acids Research 2009, 37(4), 1225-1238.

The 2′-4′ bridge comprises 2 to 4 bridging atoms and is in particular of formula —X—Y—, X being linked to C4′ and Y linked to C2′,

wherein
X is oxygen, sulfur, —CR^{a}R^{b}—, —C(R^{a})═C(R^{b})—, —C(═CR^{a}R^{b})—, —C(R^{a})═N—, —Si(R^{a})₂—, —SO₂—, —NR^{a}—; —O—NR^{a}—, —NR^{a}—O—, —C(=J)-, Se, —O—NR^{a}—, —NR^{a}—CR^{a}R^{b}—, —N(R^{a})—O— or —O—CR^{a}R^{b}—
Y is oxygen, sulfur, —(CR^{a}R^{b})ₙ—, —CR^{a}R^{b}—O—CR^{a}R^{b}—, —C(R^{a})═C(R^{b})—, —C(R^{a})═N—, —Si(R^{a})₂—, —SO₂—, —NIR^{a}—, —C(=J)-, Se, —O—NR^{a}—, —NR^{a}—CR^{a}R^{b}—, —N(R^{a})—O— or —O—CR^{a}R^{b}—;
with the proviso that —X—Y— is not —O—O—, Si(R^{a})₂—Si(R^{a})₂—, —SO₂—SO₂—, —C(R^{a})═C(R^{b})—C(R^{a})═C(R^{b}), —C(R^{a})═N—C(R^{a})═N—, —C(R^{a})═N—C(R^{a})═C(R^{b}), —C(R^{a})═C(R^{b})—C(R^{a})═N— or —Se—Se—;
J is oxygen, sulfur, ═CH₂ or ═N(R^{a});
R^{a} and R^{b} are independently selected from hydrogen, halogen, hydroxyl, cyano, thiohydroxyl, alkyl, substituted alkyl, alkenyl, substituted alkenyl, alkynyl, substituted alkynyl, alkoxy, substituted alkoxy, alkoxyalkyl, alkenyloxy, carboxyl, alkoxycarbonyl, alkylcarbonyl, formyl, aryl, heterocyclyl, amino, alkylamino, carbamoyl, alkylaminocarbonyl, aminoalkylaminocarbonyl, alkylaminoalkylaminocarbonyl, alkylcarbonylamino, carbamido, alkanoyloxy, sulfonyl, alkylsulfonyloxy, nitro, azido, thiohydroxylsulfidealkylsulfanyl, aryloxycarbonyl, aryloxy, arylcarbonyl, heteroaryl, heteroaryloxycarbonyl, heteroaryloxy, heteroarylcarbonyl, —OC(═X^{a})R^{c}, —OC(═X^{a})NR^{c}R^{d} and —NR^{e}C(═X^{a})NR^{c}R^{d};
or two geminal R^{a} and R^{b} together form optionally substituted methylene;
or two geminal R^{a} and R^{b}, together with the carbon atom to which they are attached, form cycloalkyl or halocycloalkyl, with only one carbon atom of —X—Y—;
wherein substituted alkyl, substituted alkenyl, substituted alkynyl, substituted alkoxy and substituted methylene are alkyl, alkenyl, alkynyl and methylene substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl, formyl, heterocyclyl, aryl and heteroaryl;
X^{a} is oxygen, sulfur or —NR^{c};
R^{c}, R^{d} and Re are independently selected from hydrogen and alkyl; and
n is 1, 2 or 3.

In a further particular embodiment of the invention, X is oxygen, sulfur, —NR^{a}—, —CR^{a}R^{b}— or —C(═CR^{a}R^{b})—, particularly oxygen, sulfur, —NH—, —CH₂— or —C(═CH₂)—, more particularly oxygen.

In another particular embodiment of the invention, Y is —CR^{a}R^{b}—, —CR^{a}R^{b}—CR^{a}R^{b}— or —CR^{a}R^{b}—CR^{a}R^{b}—CR^{a}R^{b}—, particularly —CH₂—CHCH₃—, —CHCH₃—CH₂—, —CH₂—CH₂— or —CH₂—CH₂—CH₂-.

In a particular embodiment of the invention, —X—Y— is —O—(CR^{a}R^{b})ₙ—, —S—CR^{a}R^{b}—, —N(R^{a})CR^{a}R^{b}—, —CR^{a}R^{b}—CR^{a}R^{b}—, —O—CR^{a}R^{b}—O—CR^{a}R^{b}—, —CR^{a}R^{b}—O—CR^{a}R^{b}—, —C(═CR^{a}R^{b})—CR^{a}R^{b}—, —N(R^{a})CR^{a}R^{b}—, —O—N(R^{a})—CR^{a}R^{b}— or —N(R^{a})—O—CR^{a}R^{b}—.

In a particular embodiment of the invention, R^{a} and R^{b} are independently selected from the group consisting of hydrogen, halogen, hydroxyl, alkyl and alkoxyalkyl, in particular hydrogen, halogen, alkyl and alkoxyalkyl.

In another embodiment of the invention, R^{a} and R^{b} are independently selected from the group consisting of hydrogen, fluoro, hydroxyl, methyl and —CH₂—O—CH₃, in particular hydrogen, fluoro, methyl and —CH₂—O—CH₃.

Advantageously, one of R^{a} and R^{b} of —X—Y— is as defined above and the other ones are all hydrogen at the same time.

In a further particular embodiment of the invention, R^{a} is hydrogen or alkyl, in particular hydrogen or methyl.

In another particular embodiment of the invention, R^{b} is hydrogen or or alkyl, in particular hydrogen or methyl.

In a particular embodiment of the invention, one or both of R^{a} and R^{b} are hydrogen.

In a particular embodiment of the invention, only one of R^{a} and R^{b} is hydrogen.

In one particular embodiment of the invention, one of R^{a} and R^{b} is methyl and the other one is hydrogen.

In a particular embodiment of the invention, R^{a} and R^{b} are both methyl at the same time.

In a particular embodiment of the invention, —X—Y— is —O—CH₂—, —S—CH₂—, —S—CH(CH₃)—, —NH—CH₂—, —O—CH₂CH₂—, —O—CH(CH₂—O—CH₃)—, —O—CH(CH₂CH₃)—, —O—CH(CH₃)—, —O—CH₂—O—CH₂—, —O—CH₂—O—CH₂—, —CH₂—O—CH₂—, —C(═CH₂)CH₂—, —C(═CH₂)CH(CH₃)—, —N(OCH₃)CH₂- or —N(CH₃)CH₂—;

In a particular embodiment of the invention, —X—Y— is —O—CR^{a}R^{b}— wherein R^{a} and R^{b} are independently selected from the group consisting of hydrogen, alkyl and alkoxyalkyl, in particular hydrogen, methyl and —CH₂—O—CH₃.

In a particular embodiment, —X—Y— is —O—CH₂— or —O—CH(CH₃)—, particularly —O—CH₂-.

The 2′-4′ bridge may be positioned either below the plane of the ribose ring (beta-D-configuration), or above the plane of the ring (alpha-L-configuration), as illustrated in formula (A) and formula (B) respectively.

The LNA nucleoside according to the invention is in particular of formula (A) or (B)

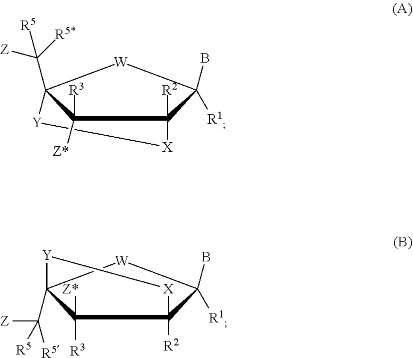

wherein
W is oxygen, sulfur, —N(R^{a})— or —CR^{a}R^{b}—, in particular oxygen;
B is a nucleobase or a modified nucleobase;
Z is an internucleoside linkage to an adjacent nucleoside or a 5′-terminal group;
Z* is an internucleoside linkage to an adjacent nucleoside or a 3′-terminal group;
R¹, R², R³, R⁵ and R^{5*} are independently selected from hydrogen, halogen, alkyl, haloalkyl, alkenyl, alkynyl, hydroxy, alkoxy, alkoxyalkyl, azido, alkenyloxy, carboxyl, alkoxycarbonyl, alkylcarbonyl, formyl and aryl; and
X, Y, R^{a} and R^{b} are as defined above.

In a particular embodiment, in the definition of —X—Y—, R^{a} is hydrogen or alkyl, in particular hydrogen or methyl. In another particular embodiment, in the definition of —X—Y—, R^{b} is hydrogen or alkyl, in particular hydrogen or methyl. In a further particular embodiment, in the definition of —X—Y—, one or both of R^{a} and R^{b} are hydrogen. In a particular embodiment, in the definition of —X—Y—, only one of R^{a} and R^{b} is hydrogen. In one particular embodiment, in the definition of —X—Y—, one of R^{a} and R^{b} is methyl and the other one is hydrogen. In a particular embodiment, in the definition of —X—Y—, R^{a} and R^{b} are both methyl at the same time.

In a further particular embodiment, in the definition of X, R^{a} is hydrogen or alkyl, in particular hydrogen or methyl. In another particular embodiment, in the definition of X, R^{b} is hydrogen or alkyl, in particular hydrogen or methyl. In a particular embodiment, in the definition of X, one or both of R^{a} and R^{b} are hydrogen. In a particular embodiment, in the definition of X, only one of R^{a} and R^{b} is hydrogen. In one particular embodiment, in the definition of X, one of R^{a} and R^{b} is methyl and the other one is hydrogen. In a particular embodiment, in the definition of X, R^{a} and R^{b} are both methyl at the same time.

In a further particular embodiment, in the definition of Y, R^{a} is hydrogen or alkyl, in particular hydrogen or methyl. In another particular embodiment, in the definition of Y, R^{b} is hydrogen or alkyl, in particular hydrogen or methyl. In a particular embodiment, in the definition of Y, one or both of R^{a} and R^{b} are hydrogen. In a particular embodiment, in the definition of Y, only one of R^{a} and R^{b} is hydrogen. In one particular embodiment, in the definition of Y, one of R^{a} and R^{b} is methyl and the other one is hydrogen. In a particular embodiment, in the definition of Y, R^{a} and R^{b} are both methyl at the same time.

In a particular embodiment of the invention R¹, R², R³, R⁵ and R^{5*} are independently selected from hydrogen and alkyl, in particular hydrogen and methyl.

In a further particular advantageous embodiment of the invention, R¹, R², R³, R⁵ and R^{5*} are all hydrogen at the same time.

In another particular embodiment of the invention, R¹, R², R³, are all hydrogen at the same time, one of R⁵ and R^{5*} is hydrogen and the other one is as defined above, in particular alkyl, more particularly methyl.

In a particular embodiment of the invention, R⁵ and R^{5*} are independently selected from hydrogen, halogen, alkyl, alkoxyalkyl and azido, in particular from hydrogen, fluoro, methyl, methoxyethyl and azido. In particular advantageous embodiments of the invention, one of R⁵ and R^{5*} is hydrogen and the other one is alkyl, in particular methyl, halogen, in particular fluoro, alkoxyalkyl, in particular methoxyethyl or azido; or R⁵ and R^{5*} are both hydrogen or halogen at the same time, in particular both hydrogen of fluoro at the same time. In such particular embodiments, W can advantageously be oxygen, and —X—Y— advantageously —O—CH₂-.

In a particular embodiment of the invention, —X—Y— is —O—CH₂—, W is oxygen and R¹, R², R³, R⁵ and R^{5*} are all hydrogen at the same time. Such LNA nucleosides are disclosed in WO 99/014226, WO 00/66604, WO 98/039352 and WO 2004/046160 which are all hereby incorporated by reference, and include what are commonly known in the art as beta-D-oxy LNA and alpha-L-oxy LNA nucleosides.

In another particular embodiment of the invention, —X—Y— is —S—CH₂—, W is oxygen and R¹, R², R³, R⁵ and R^{5*} are all hydrogen at the same time. Such thio LNA nucleosides are disclosed in WO 99/014226 and WO 2004/046160 which are hereby incorporated by reference.

In another particular embodiment of the invention, —X—Y— is —NH—CH₂—, W is oxygen and R¹, R², R³, R⁵ and R^{5*} are all hydrogen at the same time. Such amino LNA nucleosides are disclosed in WO 99/014226 and WO 2004/046160 which are hereby incorporated by reference.

In another particular embodiment of the invention, —X—Y— is —O—CH₂CH₂- or —OCH₂CH₂CH₂—, W is oxygen, and R¹, R², R³, R⁵ and R^{5*} are all hydrogen at the same time. Such LNA nucleosides are disclosed in WO 00/047599 and Morita et al., Bioorganic & Med. Chem. Lett. 12, 73-76, which are hereby incorporated by reference, and include what are commonly known in the art as 2′-O-4′C-ethylene bridged nucleic acids (ENA).

In another particular embodiment of the invention, —X—Y— is —O—CH₂—, W is oxygen, R¹, R², R³ are all hydrogen at the same time, one of R⁵ and R^{5*} is hydrogen and the other one is not hydrogen, such as alkyl, for example methyl. Such 5′ substituted LNA nucleosides are disclosed in WO 2007/134181 which is hereby incorporated by reference.

In another particular embodiment of the invention, —X—Y— is —O—CR^{a}R^{b}—, wherein one or both of R^{a} and R^{b} are not hydrogen, in particular alkyl such as methyl, W is oxygen, R¹, R², R³ are all hydrogen at the same time, one of R⁵ and R^{5*} is hydrogen and the other one is not hydrogen, in particular alkyl, for example methyl. Such bis modified LNA nucleosides are disclosed in WO 2010/077578 which is hereby incorporated by reference.

In another particular embodiment of the invention, —X—Y— is —O—CHR^{a}—, W is oxygen and R¹, R², R³, R⁵ and R^{5*} are all hydrogen at the same time. Such 6′-substituted LNA nucleosides are disclosed in WO 2010/036698 and WO 2007/090071 which are both hereby incorporated by reference. In such 6′-substituted LNA nucleosides, R^{a} is in particular C₁-C₆ alkyl, such as methyl.

In another particular embodiment of the invention, —X—Y— is —O—CH(CH₂—O—CH₃)— (“2′O-methoxyethyl bicyclic nucleic acid”, Seth et al. J. Org. Chem. 2010, Vol 75(5) pp. 1569-81).

In another particular embodiment of the invention, —X—Y— is —O—CH(CH₂CH₃)— (“2′O-ethyl bicyclic nucleic acid”, Seth at al., J. Org. Chem. 2010, Vol 75(5) pp. 1569-81).

In another particular embodiment of the invention, —X—Y— is —O—CH(CH₂—O—CH₃)—, W is oxygen and R¹, R², R³, R⁵ and R^{5*} are all hydrogen at the same time. Such LNA nucleosides are also known in the art as cyclic MOEs (cMOE) and are disclosed in WO 2007/090071.

In another particular embodiment of the invention, —X—Y— is —O—CH(CH₃)—.

In another particular embodiment of the invention, —X—Y— is —O—CH₂—O—CH₂— (Seth et al., J. Org. Chem 2010 op. cit.)

In another particular embodiment of the invention, —X—Y— is —O—CH(CH₃)—, W is oxygen and R¹, R², R³, R⁵ and R^{5*} are all hydrogen at the same time. Such 6′-methyl LNA nucleosides are also known in the art as cET nucleosides, and may be either (S)-cET or (R)-cET diastereoisomers, as disclosed in WO 2007/090071 (beta-D) and WO 2010/036698 (alpha-L) which are both hereby incorporated by reference.

In another particular embodiment of the invention, —X—Y— is —O—CR^{a}R^{b}—, wherein neither R^{a} nor R^{b} is hydrogen, W is oxygen and R¹, R², R³, R⁵ and R^{5*} are all hydrogen at the same time. In a particular embodiment, R^{a} and R^{b} are both alkyl at the same time, in particular both methyl at the same time. Such 6′-di-substituted LNA nucleosides are disclosed in WO 2009/006478 which is hereby incorporated by reference.

In another particular embodiment of the invention, —X—Y— is —S—CHIR^{a}—, W is oxygen and R¹, R², R³, R⁵ and R^{5*} are all hydrogen at the same time. Such 6′-substituted thio LNA nucleosides are disclosed in WO 2011/156202 which is hereby incorporated by reference. In a particular embodiment of such 6′-substituted thio LNA, R^{a} is alkyl, in particular methyl.

In a particular embodiment of the invention, —X—Y— is —C(═CH₂)C(R^{a}R^{b})—, —C(═CHF)C(R^{a}R^{b})— or —C(═CF₂)C(R^{a}R^{b})—, W is oxygen and R¹, R², R³, R⁵ and R^{5*} are all hydrogen at the same time. R^{a} and R^{b} are advantageously independently selected from hydrogen, halogen, alkyl and alkoxyalkyl, in particular hydrogen, methyl, fluoro and methoxymethyl. R^{a} and R^{b} are in particular both hydrogen or methyl at the same time or one of R^{a} and R^{b} is hydrogen and the other one is methyl. Such vinyl carbo LNA nucleosides are disclosed in WO 2008/154401 and WO 2009/067647 which are both hereby incorporated by reference.

In a particular embodiment of the invention, —X—Y— is-N(OR^{a})—CH₂—, W is oxygen and R¹, R², R³, R⁵ and R^{5*} are all hydrogen at the same time. In a particular embodiment, R^{a} is alkyl such as methyl. Such LNA nucleosides are also known as N substituted LNAs and are disclosed in WO 2008/150729 which is hereby incorporated by reference.

In a particular embodiment of the invention, —X—Y— is —O—N(R^{a})—, —N(R^{a})—O—, —NR^{a}—CR^{a}R^{b}—CR^{a}R^{b}— or —NR^{a}—CR^{a}R^{b}—, W is oxygen and R¹, R², R³, R⁵ and R^{5*} are all hydrogen at the same time. R^{a} and R^{b} are advantageously independently selected from hydrogen, halogen, alkyl and alkoxyalkyl, in particular hydrogen, methyl, fluoro and methoxymethyl. In a particular embodiment, R^{a} is alkyl, such as methyl, R^{b} is hydrogen or methyl, in particular hydrogen. (Seth et al., J. Org. Chem 2010 op. cit.).

In a particular embodiment of the invention, —X—Y— is —O—N(CH₃)— (Seth et al., J. Org. Chem 2010 op. cit.).

In a particular embodiment of the invention, R⁵ and R^{5*} are both hydrogen at the same time. In another particular embodiment of the invention, one of R⁵ and R^{5*} is hydrogen and the other one is alkyl, such as methyl. In such embodiments, R¹, R² and R³ can be in particular hydrogen and —X—Y— can be in particular —O—CH₂— or —O—CHC(R^{a})₃—, such as —O—CH(CH₃)—.

In a particular embodiment of the invention, —X—Y— is —CR^{a}R^{b}—O—CR^{a}R^{b}—, such as —CH₂—O—CH₂—, W is oxygen and R¹, R², R³, R⁵ and R^{5*} are all hydrogen at the same time. In such particular embodiments, R^{a} can be in particular alkyl such as methyl, R^{b} hydrogen or methyl, in particular hydrogen. Such LNA nucleosides are also known as conformationally restricted nucleotides (CRNs) and are disclosed in WO 2013/036868 which is hereby incorporated by reference.

In a particular embodiment of the invention, —X—Y— is-O—CR^{a}R^{b}—O—CR^{a}R^{b}—, such as —O—CH₂—O—CH₂—, W is oxygen and R¹, R², R³, R⁵ and R^{5*} are all hydrogen at the same time. R^{a} and R^{b} are advantageously independently selected from hydrogen, halogen, alkyl and alkoxyalkyl, in particular hydrogen, methyl, fluoro and methoxymethyl. In such a particular embodiment, R^{a} can be in particular alkyl such as methyl, R^{b} hydrogen or methyl, in particular hydrogen. Such LNA nucleosides are also known as COC nucleotides and are disclosed in Mitsuoka et al., Nucleic Acids Research 2009, 37(4), 1225-1238, which is hereby incorporated by reference.

It will be recognized than, unless specified, the LNA nucleosides may be in the beta-D or alpha-L stereoisoform.

Particular examples of LNA nucleosides of the invention are presented in Scheme 1 (wherein B is as defined above).

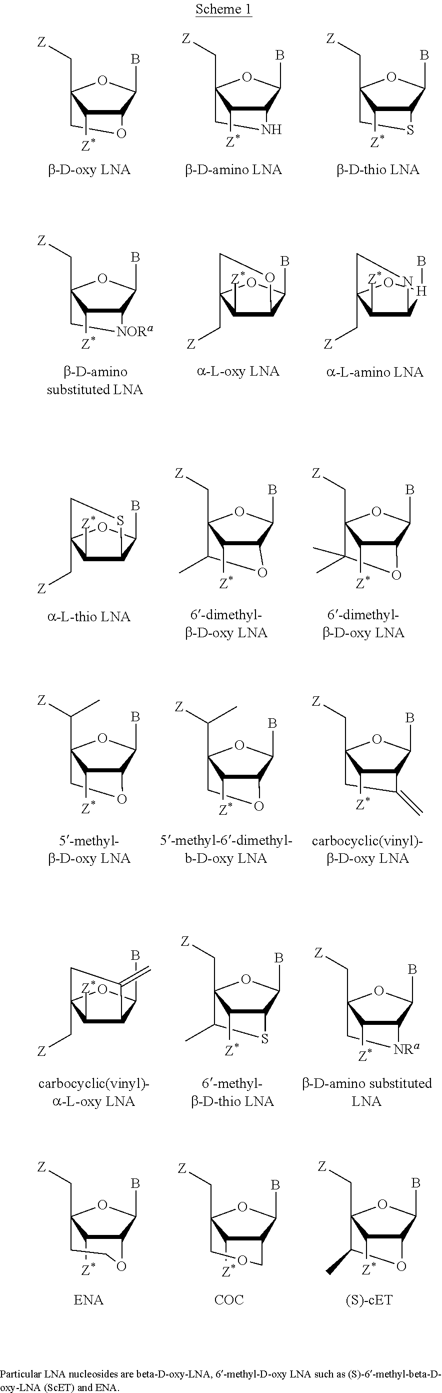

### Chemical Group Definitions

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 4 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl. Particular examples of alkyl are methyl, ethyl and propyl.

The term “cycloalkyl”, alone or in combination, signifies a cycloalkyl ring with 3 to 8 carbon atoms and particularly a cycloalkyl ring with 3 to 6 carbon atoms. Examples of cycloalkyl are cyclopropyl, cyclobutyl, cyclopentyl, cyclohexyl, cycloheptyl and cyclooctyl, more particularly cyclopropyl and cyclobutyl. A particular example of “cycloalkyl” is cyclopropyl.

The term “alkoxy”, alone or in combination, signifies a group of the formula alkyl-O— in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy and ethoxy. Methoxyethoxy is a particular example of “alkoxyalkoxy”.

The term “oxy”, alone or in combination, signifies the —O— group.

The term “alkenyl”, alone or in combination, signifies a straight-chain or branched hydrocarbon residue comprising an olefinic bond and up to 8, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl and isobutenyl.

The term “alkynyl”, alone or in combination, signifies a straight-chain or branched hydrocarbon residue comprising a triple bond and up to 8, preferably up to 6, particularly preferred up to 4 carbon atoms.

The terms “halogen” or “halo”, alone or in combination, signifies fluorine, chlorine, bromine or iodine and particularly fluorine, chlorine or bromine, more particularly fluorine. The term “halo”, in combination with another group, denotes the substitution of said group with at least one halogen, particularly substituted with one to five halogens, particularly one to four halogens, i.e. one, two, three or four halogens.

The term “haloalkyl”, alone or in combination, denotes an alkyl group substituted with at least one halogen, particularly substituted with one to five halogens, particularly one to three halogens. Examples of haloalkyl include monofluoro-, difluoro- or trifluoro-methyl, -ethyl or -propyl, for example 3,3,3-trifluoropropyl, 2-fluoroethyl, 2,2,2-trifluoroethyl, fluoromethyl or trifluoromethyl. Fluoromethyl, difluoromethyl and trifluoromethyl are particular “haloalkyl”.

The term “halocycloalkyl”, alone or in combination, denotes a cycloalkyl group as defined above substituted with at least one halogen, particularly substituted with one to five halogens, particularly one to three halogens. Particular example of “halocycloalkyl” are halocyclopropyl, in particular fluorocyclopropyl, difluorocyclopropyl and trifluorocyclopropyl.

The terms “hydroxyl” and “hydroxy”, alone or in combination, signify the —OH group.

The terms “thiohydroxyl” and “thiohydroxy”, alone or in combination, signify the —SH group.

The term “carbonyl”, alone or in combination, signifies the —C(O)— group.

The term “carboxy” or “carboxyl”, alone or in combination, signifies the —COOH group.

The term “amino”, alone or in combination, signifies the primary amino group (—NH₂), the secondary amino group (—NH—), or the tertiary amino group (—N—).

The term “alkylamino”, alone or in combination, signifies an amino group as defined above substituted with one or two alkyl groups as defined above.

The term “sulfonyl”, alone or in combination, means the —SO₂ group.

The term “sulfinyl”, alone or in combination, signifies the —SO— group.

The term “sulfanyl”, alone or in combination, signifies the —S— group.

The term “cyano”, alone or in combination, signifies the —CN group.

The term “azido”, alone or in combination, signifies the —N₃ group.

The term “nitro”, alone or in combination, signifies the NO₂ group.

The term “formyl”, alone or in combination, signifies the —C(O)H group.

The term “carbamoyl”, alone or in combination, signifies the —C(O)NH₂ group.

The term “cabamido”, alone or in combination, signifies the —NH—C(O)—NH₂ group.

The term “aryl”, alone or in combination, denotes a monovalent aromatic carbocyclic mono- or bicyclic ring system comprising 6 to 10 carbon ring atoms, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of aryl include phenyl and naphthyl, in particular phenyl.

The term “heteroaryl”, alone or in combination, denotes a monovalent aromatic heterocyclic mono- or bicyclic ring system of 5 to 12 ring atoms, comprising 1, 2, 3 or 4 heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples of heteroaryl include pyrrolyl, furanyl, thienyl, imidazolyl, oxazolyl, thiazolyl, triazolyl, oxadiazolyl, thiadiazolyl, tetrazolyl, pyridinyl, pyrazinyl, pyrazolyl, pyridazinyl, pyrimidinyl, triazinyl, azepinyl, diazepinyl, isoxazolyl, benzofuranyl, isothiazolyl, benzothienyl, indolyl, isoindolyl, isobenzofuranyl, benzimidazolyl, benzoxazolyl, benzoisoxazolyl, benzothiazolyl, benzoisothiazolyl, benzooxadiazolyl, benzothiadiazolyl, benzotriazolyl, purinyl, quinolinyl, isoquinolinyl, quinazolinyl, quinoxalinyl, carbazolyl or acridinyl.

The term “heterocyclyl”, alone or in combination, signifies a monovalent saturated or partly unsaturated mono- or bicyclic ring system of 4 to 12, in particular 4 to 9 ring atoms, comprising 1, 2, 3 or 4 ring heteroatoms selected from N, O and S, the remaining ring atoms being carbon, optionally substituted with 1 to 3 substituents independently selected from halogen, hydroxyl, alkyl, alkenyl, alkynyl, alkoxy, alkoxyalkyl, alkenyloxy, carboxyl, alkoxycarbonyl, alkylcarbonyl and formyl. Examples for monocyclic saturated heterocyclyl are azetidinyl, pyrrolidinyl, tetrahydrofuranyl, tetrahydro-thienyl, pyrazolidinyl, imidazolidinyl, oxazolidinyl, isoxazolidinyl, thiazolidinyl, piperidinyl, tetrahydropyranyl, tetrahydrothiopyranyl, piperazinyl, morpholinyl, thiomorpholinyl, 1,1-dioxo-thiomorpholin-4-yl, azepanyl, diazepanyl, homopiperazinyl, or oxazepanyl. Examples for bicyclic saturated heterocycloalkyl are 8-aza-bicyclo[3.2.1]octyl, quinuclidinyl, 8-oxa-3-aza-bicyclo[3.2.1]octyl, 9-aza-bicyclo[3.3.1]nonyl, 3-oxa-9-aza-bicyclo[3.3.1]nonyl, or 3-thia-9-aza-bicyclo[3.3.1]nonyl. Examples for partly unsaturated heterocycloalkyl are dihydrofuryl, imidazolinyl, dihydro-oxazolyl, tetrahydro-pyridinyl or dihydropyranyl.

Pharmaceutically Acceptable Salts

The term “pharmaceutically acceptable salts” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid, particularly hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein. In addition these salts may be prepared form addition of an inorganic base or an organic base to the free acid. Salts derived from an inorganic base include, but are not limited to, the sodium, potassium, lithium, ammonium, calcium, magnesium salts. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyamine resins. The compound of formula (I) can also be present in the form of zwitterions. Particularly preferred pharmaceutically acceptable salts of compounds of formula (I) are the salts of hydrochloric acid, hydrobromic acid, sulfuric acid, phosphoric acid and methanesulfonic acid.

Protecting Group

The term “protecting group”, alone or in combination, signifies a group which selectively blocks a reactive site in a multifunctional compound such that a chemical reaction can be carried out selectively at another unprotected reactive site. Protecting groups can be removed. Exemplary protecting groups are amino-protecting groups, carboxy-protecting groups or hydroxy-protecting groups.

Nuclease Mediated Degradation

Nuclease mediated degradation refers to an oligonucleotide capable of mediating degradation of a complementary nucleotide sequence when forming a duplex with such a sequence.

In some embodiments, the oligonucleotide may function via nuclease mediated degradation of the target nucleic acid, where the oligonucleotides of the invention are capable of recruiting a nuclease, particularly and endonuclease, preferably endoribonuclease (RNase), such as RNase H. Examples of oligonucleotide designs which operate via nuclease mediated mechanisms are oligonucleotides which typically comprise a region of at least 5 or 6 DNA nucleosides and are flanked on one side or both sides by affinity enhancing nucleosides, for example gapmers, headmers and tailmers.

RNase H Activity and Recruitment

The RNase H activity of an antisense oligonucleotide refers to its ability to recruit RNase H when in a duplex with a complementary RNA molecule. WO01/23613 provides in vitro methods for determining RNaseH activity, which may be used to determine the ability to recruit RNaseH. Typically an oligonucleotide is deemed capable of recruiting RNase H if it, when provided with a complementary target nucleic acid sequence, has an initial rate, as measured in pmol/l/min, of at least 5%, such as at least 10% or more than 20% of the of the initial rate determined when using a oligonucleotide having the same base sequence as the modified oligonucleotide being tested, but containing only DNA monomers with phosphorothioate linkages between all monomers in the oligonucleotide, and using the methodology provided by Example 91-95 of WO01/23613 (hereby incorporated by reference). For use in determining RHase H activity, recombinant human RNase H1 is available from Lubio Science GmbH, Lucerne, Switzerland.

Gapmer

The antisense oligonucleotide of the invention, or contiguous nucleotide sequence thereof may be a gapmer. The antisense gapmers are commonly used to inhibit a target nucleic acid via RNase H mediated degradation. A gapmer oligonucleotide comprises at least three distinct structural regions a 5′-flank, a gap and a 3′-flank, F-G-F′ in the ‘5->3’ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, advantageously high affinity sugar modified nucleosides, and by a 3′ flanking region (F′) comprising one or more sugar modified nucleosides, advantageously high affinity sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as high affinity 2′ sugar modifications, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further defined by having at least one sugar modified nucleoside at the end most distant from the gap region, i.e. at the 5′ end of the 5′ flank and at the 3′ end of the 3′ flank.

Regions F-G-F′ form a contiguous nucleotide sequence. Antisense oligonucleotides of the invention, or the contiguous nucleotide sequence thereof, may comprise a gapmer region of formula F-G-F′.

The overall length of the gapmer design F-G-F′ may be, for example 12 to 32 nucleosides, such as 13 to 24, such as 14 to 22 nucleosides, Such as from 14 to 17, such as 16 to 18 nucleosides.

By way of example, the gapmer oligonucleotide of the present invention can be represented by the following formulae:

F₁₋₈-G₅₋₁₆-F′₁₋₈, such as

F₁₋₈-G₇₋₁₆-F₂₋₈

with the proviso that the overall length of the gapmer regions F-G-F′ is at least 12, such as at least 14 nucleotides in length.

Regions F, G and F′ are further defined below and can be incorporated into the F-G-F′ formula.

Gapmer-Region G

Region G (gap region) of the gapmer is a region of nucleosides which enables the oligonucleotide to recruit RNaseH, such as human RNase H1, typically DNA nucleosides. RNaseH is a cellular enzyme which recognizes the duplex between DNA and RNA, and enzymatically cleaves the RNA molecule. Suitably gapmers may have a gap region (G) of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. Cytosine (C) DNA in the gap region may in some instances be methylated, such residues are either annotated as 5-methyl-cytosine (meC or with an e instead of a c). Methylation of Cytosine DNA in the gap is advantageous if cg dinucleotides are present in the gap to reduce potential toxicity, the modification does not have significant impact on efficacy of the oligonucleotides.

In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages.

Whilst traditional gapmers have a DNA gap region, there are numerous examples of modified nucleosides which allow for RNaseH recruitment when they are used within the gap region. Modified nucleosides which have been reported as being capable of recruiting RNaseH when included within a gap region include, for example, alpha-L-LNA, C4′ alkylated DNA (as described in PCT/EP2009/050349 and Vester et al., Bioorg. Med. Chem. Lett. 18 (2008) 2296-2300, both incorporated herein by reference), arabinose derived nucleosides like ANA and 2′F-ANA (Mangos et al. 2003 J. AM. CHEM. SOC. 125, 654-661), UNA (unlocked nucleic acid) (as described in Fluiter et al., Mol. Biosyst., 2009, 10, 1039 incorporated herein by reference). UNA is unlocked nucleic acid, typically where the bond between C2 and C3 of the ribose has been removed, forming an unlocked “sugar” residue. The modified nucleosides used in such gapmers may be nucleosides which adopt a 2′ endo (DNA like) structure when introduced into the gap region, i.e. modifications which allow for RNaseH recruitment). In some embodiments the DNA Gap region (G) described herein may optionally contain 1 to 3 sugar modified nucleosides which adopt a 2′ endo (DNA like) structure when introduced into the gap region.

Region G-“Gap-Breaker”

Alternatively, there are numerous reports of the insertion of a modified nucleoside which confers a 3′ endo conformation into the gap region of gapmers, whilst retaining some RNaseH activity. Such gapmers with a gap region comprising one or more 3′endo modified nucleosides are referred to as “gap-breaker” or “gap-disrupted” gapmers, see for example WO2013/022984. Gap-breaker oligonucleotides retain sufficient region of DNA nucleosides within the gap region to allow for RNaseH recruitment. The ability of gapbreaker oligonucleotide design to recruit RNaseH is typically sequence or even compound specific —see Rukov et al. 2015 Nucl. Acids Res. Vol. 43 pp. 8476-8487, which discloses “gapbreaker” oligonucleotides which recruit RNaseH which in some instances provide a more specific cleavage of the target RNA. Modified nucleosides used within the gap region of gap-breaker oligonucleotides may for example be modified nucleosides which confer a 3′endo confirmation, such 2′-O-methyl (OMe) or 2′-O-MOE (MOE) nucleosides, or beta-D LNA nucleosides (the bridge between C2′ and C4′ of the ribose sugar ring of a nucleoside is in the beta conformation), such as beta-D-oxy LNA or ScET nucleosides.

As with gapmers containing region G described above, the gap region of gap-breaker or gap-disrupted gapmers, have a DNA nucleosides at the 5′ end of the gap (adjacent to the 3′ nucleoside of region F), and a DNA nucleoside at the 3′ end of the gap (adjacent to the 5′ nucleoside of region F′). Gapmers which comprise a disrupted gap typically retain a region of at least 3 or 4 contiguous DNA nucleosides at either the 5′ end or 3′ end of the gap region.

Exemplary designs for gap-breaker oligonucleotides include

F₁₋₈-[D₃₋₄-E₁-D₃₋₄]-F′₁₋₈

F₁₋₈-[D₁₋₄-E₁-D₃₋₄]-F′₁₋₈

F₁₋₈-[D₃₋₄-E₁-D₁₋₄]-F′₁₋₈

wherein region G is within the brackets [Dₙ-Eᵣ-Dₘ], D is a contiguous sequence of DNA nucleosides, E is a modified nucleoside (the gap-breaker or gap-disrupting nucleoside), and F and F′ are the flanking regions as defined herein, and with the proviso that the overall length of the gapmer regions F-G-F′ is at least 12, such as at least 14 nucleotides in length.

In some embodiments, region G of a gap disrupted gapmer comprises at least 6 DNA nucleosides, such as 6, 7, 8, 9, 10, 11, 12, 13, 14, 15 or 16 DNA nucleosides. As described above, the DNA nucleosides may be contiguous or may optionally be interspersed with one or more modified nucleosides, with the proviso that the gap region G is capable of mediating RNaseH recruitment.

Gapmer-Flanking Regions, F and F′

Region F is positioned immediately adjacent to the 5′ DNA nucleoside of region G. The 3′ most nucleoside of region F is a sugar modified nucleoside, such as a high affinity sugar modified nucleoside, for example a 2′ substituted nucleoside, such as a MOE nucleoside, or an LNA nucleoside.

Region F′ is positioned immediately adjacent to the 3′ DNA nucleoside of region G. The 5′ most nucleoside of region F′ is a sugar modified nucleoside, such as a high affinity sugar modified nucleoside, for example a 2′ substituted nucleoside, such as a MOE nucleoside, or an LNA nucleoside.

Region F is 1-8 contiguous nucleotides in length, such as 2-6, such as 3-4 contiguous nucleotides in length. Advantageously the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

Region F′ is 2-8 contiguous nucleotides in length, such as 3-6, such as 4-5 contiguous nucleotides in length. Advantageously, embodiments the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is advantageously an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units.

In some embodiments, region F and F′ independently comprises both LNA and a 2′ substituted modified nucleosides (mixed wing design).

In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are beta-D-oxy LNA nucleosides.

In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments, all the modified nucleosides of region F and F′ are LNA nucleosides, such as independently selected from beta-D-oxy LNA, ENA or ScET nucleosides, wherein region F or F′, or F and F′ may optionally comprise DNA nucleosides (an alternating flank, see definition of these for more details). In some embodiments, all the modified nucleosides of region F and F′ are beta-D-oxy LNA nucleosides, wherein region F or F′, or F and F′ may optionally comprise DNA nucleosides (an alternating flank, see definition of these for more details).

In some embodiments the 5′ most and the 3′ most nucleosides of region F and F′ are LNA nucleosides, such as beta-D-oxy LNA nucleosides or ScET nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

Further gapmer designs are disclosed in WO 2004/046160, WO 2007/146511 and WO 2008/113832, hereby incorporated by reference.

LNA Gapmer

An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In some embodiments the LNA gapmer is of formula: [LNA]₁₋₆-[region G]-[LNA]₁₋₆, wherein region G is as defined in the Gapmer region G definition.

MOE Gapmers

A MOE gapmers is a gapmer wherein regions F and F′ consist of MOE nucleosides. In some embodiments the MOE gapmer is of design [MOE]₁₋₈-[Region G]-[MOE]₁₋₈, such as [MOE]₂₋₇-[Region G]₆₋₁₆-[MOE]₂₋₇, such as [MOE]₃₋₆-[Region G]-[MOE]₃₋₈, wherein region G is as defined in the Gapmer definition. MOE gapmers with a 5-10-5 design (MOE-DNA-MOE) have been widely used in the art.

Mixed Wing Gapmer

A mixed wing gapmer is an LNA gapmer wherein one or both of region F and F′ comprise a 2′ substituted nucleoside, such as a 2′ substituted nucleoside independently selected from the group consisting of 2′-O-alkyl-RNA units, 2′-O-methyl-RNA, 2′-amino-DNA units, 2′-fluoro-DNA units, 2′-alkoxy-RNA, MOE units, arabino nucleic acid (ANA) units and 2′-fluoro-ANA units, such as a MOE nucleosides. In some embodiments wherein at least one of region F and F′, or both region F and F′ comprise at least one LNA nucleoside, the remaining nucleosides of region F and F′ are independently selected from the group consisting of MOE and LNA. In some embodiments wherein at least one of region F and F′, or both region F and F′ comprise at least two LNA nucleosides, the remaining nucleosides of region F and F′ are independently selected from the group consisting of MOE and LNA. In some mixed wing embodiments, one or both of region F and F′ may further comprise one or more DNA nucleosides.

Mixed wing gapmer designs are disclosed in WO 2008/049085 and WO 2012/109395, both of which are hereby incorporated by reference.

Alternating Flank Gapmers

Flanking regions may comprise both LNA and DNA nucleoside and are referred to as “alternating flanks” as they comprise an alternating motif of LNA-DNA-LNA nucleosides. Gapmers comprising such alternating flanks are referred to as “alternating flank gapmers”. “Alternative flank gapmers” are thus LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

Alternating flank LNA gapmers are disclosed in WO 2016/127002.

Oligonucleotides with alternating flanks are LNA gapmer oligonucleotides where at least one of the flanks (F or F′) comprises DNA in addition to the LNA nucleoside(s). In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F and/or F′ region are LNA nucleosides.

In some embodiments at least one of region F or F′, or both region F and F′, comprise both LNA nucleosides and DNA nucleosides. In such embodiments, the flanking region F or F′, or both F and F′ comprise at least three nucleosides, wherein the 5′ and 3′ most nucleosides of the F or F′ region are LNA nucleosides, and the. Flanking regions which comprise both LNA and DNA nucleoside are referred to as alternating flanks, as they comprise an alternating motif of LNA-DNA-LNA nucleosides. Alternating flank LNA gapmers are disclosed in WO2016/127002.

An alternating flank region may comprise up to 3 contiguous DNA nucleosides, such as 1 to 2 or 1 or 2 or 3 contiguous DNA nucleosides.

The alternating flak can be annotated as a series of integers, representing a number of LNA nucleosides (L) followed by a number of DNA nucleosides (D), for example

[L]₁₋₃-[D]₁₋₄-[L]₁₋₃

[L]₁₋₂-[D]₁₋₂-[L]₁₋₂-[D]₁₋₂-[L]₁₋₂

In oligonucleotide designs these will often be represented as numbers such that 2-2-1 represents 5′[L]₂-[D]₂-[L] 3′, and 1-1-1-1-1 represents 5′[L]-[D]-[L]-[D]-[L] 3′. The length of the flank (region F and F′) in oligonucleotides with alternating flanks may independently be 3 to 10 nucleosides, such as 4 to 8, such as 5 to 6 nucleosides, such as 4, 5, 6 or 7 modified nucleosides. In some embodiments only one of the flanks in the gapmer oligonucleotide is alternating while the other is constituted of LNA nucleotides. It may be advantageous to have at least two LNA nucleosides at the 3′ end of the 3′ flank (F′), to confer additional exonuclease resistance. Some examples of oligonucleotides with alternating flanks are:

[L]₁₋₅-[D]₁₋₄-[L]₁₋₃-[G]₅₋₁₆-[L]₂₋₆

[L]₁₋₂-[D]₁₋₂-[L]₁₋₂-[D]₁₋₂-[L]₁₋₂[G]₅₋₁₆-[L]₁₋₂-[D]₁₋₃-[L]₂₋₄

[L]₁₋₅-[G]₅₋₁₆-[L]-[D]-[L]-[D]-[L]₂

with the proviso that the overall length of the gapmer is at least 12, such as at least 14 nucleotides in length.

Region D′ or D″ in an Oligonucleotide

The oligonucleotide of the invention may in some embodiments comprise or consist of the contiguous nucleotide sequence of the oligonucleotide which is complementary to the target nucleic acid, such as the gapmer F-G-F′, and further 5′ and/or 3′ nucleosides. The further 5′ and/or 3′ nucleosides may or may not be fully complementary to the target nucleic acid. Such further 5′ and/or 3′ nucleosides may be referred to as region D′ and D″ herein.

The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively it may be used to provide exonucleoase protection or for ease of synthesis or manufacture.

Region D′ and D″ can be attached to the 5′ end of region F or the 3′ end of region F′, respectively to generate designs of the following formulas D′-F-G-F′, F-G-F′-D″ or

D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA. Nucleotide based biocleavable linkers suitable for use as region D′ or D″ are disclosed in WO 2014/076195, which include by way of example a phosphodiester linked DNA dinucleotide. The use of biocleavable linkers in poly-oligonucleotide constructs is disclosed in WO 2015/113922, where they are used to link multiple antisense constructs (e.g. gapmer regions) within a single oligonucleotide.

In one embodiment the oligonucleotide of the invention comprises a region D′ and/or D″ in addition to the contiguous nucleotide sequence which constitutes the gapmer.

In some embodiments, the oligonucleotide of the present invention can be represented by the following formulae:

F-G-F′; in particular F₁₋₈-G₅₋₁₆-F₂₋₈

D′-F-G-F′, in particular D′₁₋₃-F₁₋₈-G₅₋₁₆-F₂₋₈

F-G-F′-D″, in particular F₁₋₈-G₅₋₁₆-F₂₋₈-D″₁₋₃

D′-F-G-F′-D″, in particular D′₁₋₃-F₁₋₈-G₅₋₁₆-F₂₋₈-D″₁₋₃

In some embodiments the internucleoside linkage positioned between region D′ and region F is a phosphodiester linkage. In some embodiments the internucleoside linkage positioned between region F′ and region D″ is a phosphodiester linkage.

Conjugate

The term conjugate as used herein refers to an oligonucleotide which is covalently linked to a non-nucleotide moiety (conjugate moiety or region C or third region).

Conjugation of the oligonucleotide of the invention to one or more non-nucleotide moieties may improve the pharmacology of the oligonucleotide, e.g. by affecting the activity, cellular distribution, cellular uptake or stability of the oligonucleotide. In some embodiments the conjugate moiety modify or enhance the pharmacokinetic properties of the oligonucleotide by improving cellular distribution, bioavailability, metabolism, excretion, permeability, and/or cellular uptake of the oligonucleotide. In particular the conjugate may target the oligonucleotide to a specific organ, tissue or cell type and thereby enhance the effectiveness of the oligonucleotide in that organ, tissue or cell type. A the same time the conjugate may serve to reduce activity of the oligonucleotide in non-target cell types, tissues or organs, e.g. off target activity or activity in non-target cell types, tissues or organs. WO 93/07883 and WO2013/033230 provides suitable conjugate moieties, which are hereby incorporated by reference. Further suitable conjugate moieties are those capable of binding to the asialoglycoprotein receptor (ASGPr). In particular tri-valent N-acetylgalactosamine conjugate moieties are suitable for binding to the ASGPr, see for example WO 2014/076196, WO 2014/207232 and WO 2014/179620 (hereby incorporated by reference, in particular, FIG. 13 of WO2014/076196 or claims 158-164 of WO2014/179620).

Oligonucleotide conjugates and their synthesis has also been reported in comprehensive reviews by Manoharan in Antisense Drug Technology, Principles, Strategies, and Applications, S. T. Crooke, ed., Ch. 16, Marcel Dekker, Inc., 2001 and Manoharan, Antisense and Nucleic Acid Drug Development, 2002, 12, 103, each of which is incorporated herein by reference in its entirety.

In an embodiment, the non-nucleotide moiety (conjugate moiety) is selected from the group consisting of carbohydrates, cell surface receptor ligands, drug substances, hormones, lipophilic substances, polymers, proteins, peptides, toxins (e.g. bacterial toxins), vitamins, viral proteins (e.g. capsids) or combinations thereof.

Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety (Region C), to a first region, e.g. an oligonucleotide or contiguous nucleotide sequence complementary to the target nucleic acid (region A), thereby connecting one of the termini of region A to C.

In some embodiments of the invention the conjugate or oligonucleotide conjugate of the invention may optionally, comprise a linker region (second region or region B and/or region Y) which is positioned between the oligonucleotide or contiguous nucleotide sequence complementary to the target nucleic acid (region A or first region) and the conjugate moiety (region C or third region).

Region B refers to biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to 51 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Conjugates may also be linked to the oligonucleotide via non-biocleavable linkers, or in some embodiments the conjugate may comprise a non-cleavable linker which is covalently attached to the biocleavable linker (region Y). Linkers that are not necessarily biocleavable but primarily serve to covalently connect a conjugate moiety (region C or third region), to an oligonucleotide (region A or first region), may comprise a chain structure or an oligomer of repeating units such as ethylene glycol, amino acid units or amino alkyl groups The oligonucleotide conjugates of the present invention can be constructed of the following regional elements A-C, A-B-C, A-B-Y-C, A-Y-B-C or A-Y-C. In some embodiments the non-cleavable linker (region Y) is an amino alkyl, such as a C2-C36 amino alkyl group, including, for example C6 to C12 amino alkyl groups. In a preferred embodiment the linker (region Y) is a C6 amino alkyl group. Conjugate linker groups may be routinely attached to an oligonucleotide via use of an amino modified oligonucleotide, and an activated ester group on the conjugate group.

Treatment

The term ‘treatment’ as used herein refers to both treatment of an existing disease (e.g. a disease or disorder as herein referred to), or prevention of a disease, i.e. prophylaxis. It will therefore be recognized that treatment as referred to herein may, in some embodiments, be prophylactic.

### DETAILED DESCRIPTION OF THE INVENTION

The Oligonucleotides of the Invention

The invention relates to oligonucleotides capable of inhibiting expression of FNDC3B. The inhibition may be achieved by hybridizing to a target nucleic acid encoding FNDC3B or which is involved in the regulation of FNDC3B. The target nucleic acid may be a mammalian FNDC3B sequence, such as a sequence selected from the group consisting of SEQ ID NO: 1, 2, 3, 4, 5, 6, 7 and 8.

The oligonucleotide of the invention is an antisense oligonucleotide which targets FNDC3B.

In some embodiments the antisense oligonucleotide of the invention is capable of modulating the expression of the target by inhibiting or reducing target expression. Preferably, such inhibition of expression is at least 20% compared to the normal expression level of the target, more preferably at least 30%, at least 40%, at least 50%, at least 60%, at least 70%, at least 80%, at least 90% or at least 95% inhibition compared to the normal expression level of the target. In some embodiments oligonucleotides of the invention may be capable of inhibiting expression levels of FNDC3B mRNA by at least 60% or at least 70% in vitro using HeLa cells.

In some embodiments compounds of the invention may be capable of inhibiting expression levels of FNDC3B protein by at least 50% in vitro using HeLa cells. Suitably, the examples provide assays which may be used to measure FNDC3B RNA or protein inhibition (e.g. example 1). The target modulation is triggered by the hybridization between a contiguous nucleotide sequence of the oligonucleotide and the target nucleic acid. In some embodiments the oligonucleotide of the invention comprises mismatches between the oligonucleotide and the target nucleic acid. Despite mismatches hybridization to the target nucleic acid may still be sufficient to show a desired modulation of FNDC3B expression. Reduced binding affinity resulting from mismatches may advantageously be compensated by increased number of nucleotides in the oligonucleotide and/or an increased number of modified nucleosides capable of increasing the binding affinity to the target, such as 2′ sugar modified nucleosides, including LNA, present within the oligonucleotide sequence.

An aspect of the present invention relates to an antisense oligonucleotide of 10 to 50, such as 10-30, nucleotides in length, which comprises a contiguous nucleotide sequence of 10 to 30 nucleotides in length with at least 90% complementarity, such as full complementarity, to a mammalian FNDC3B encoding target nucleic acid, wherein the antisense oligonucleotide is capable of reducing the expression of the mammalian FNDC3B encoding target nucleic acid in a cell.

An aspect of the present invention relates to an antisense oligonucleotide of 10 to 30 nucleotides in length, which comprises a contiguous nucleotide sequence of 12 to 22 nucleotides in length with at least 90% complementarity, such as full complementarity, to a mammalian FNDC3B encoding target nucleic acid, wherein the antisense oligonucleotide is capable of reducing the expression of the mammalian FNDC3B encoding target nucleic acid in a cell.

In some embodiments, the oligonucleotide comprises a contiguous nucleotide sequence of 10 to 30 nucleotides in length, such as 12-22 nucleotides in length, which is at least 90% complementary, such as at least 91%, such as at least 92%, such as at least 93%, such as at least 94%, such as at least 95%, such as at least 96%, such as at least 97%, such as at least 98%, or 100% complementary with the target nucleic acid or the target sequence.

In a preferred embodiment the antisense oligonucleotide of the invention, or contiguous nucleotide sequence thereof is fully complementary (100% complementary) to the target nucleic acid or the target sequence, or in some embodiments may comprise one or two mismatches between the oligonucleotide and the target nucleic acid.

Another aspect of the present invention relates to the antisense oligonucleotide, wherein the contiguous nucleotide sequence is at least 90% complementary to a sequence selected from the group consisting of SEQ ID NO1, 2, 3, 4, 5, 6, 7 and 8, or a naturally occurring variant thereof.

In some embodiments the oligonucleotide sequence or contiguous nucleotide sequence is at least 90% complementary or at least 95% complementary such as fully complementary to a corresponding target sequence present in SEQ ID NO: 1 and SEQ ID NO: 8. In some embodiments the contiguous sequence of the antisense oligonucleotide is fully complementary to the mammalian FNDC3B encoding target nucleic acid.

In a preferred embodiment the oligonucleotide sequence or contiguous nucleotide sequence is 100% complementary to a corresponding target sequence present in SEQ ID NO: 1 and SEQ ID NO: 8.

Another aspect of the present invention relates to the antisense oligonucleotide, wherein the contiguous nucleotide sequence is at least 90% complementary, such as fully complementary, to an intron region present in the pre-mRNA of mammalian FNDC3B encoding target nucleic acid (e.g. SEQ ID NO 1).

It shall be understood that intron positions on SEQ ID NO: 1 may vary depending on different splicing of FND3B pre-mRNA. In the context of the present invention any nuclotide sequence in the gene sequence or pre-mRNA that is removed from the pre-mRNA by RNA splicing during maturation of the final RNA product (mature mRNA) are introns irrespectively on their position on SEQ ID NO: 1. Table 1 provides the most common intron regions in SEQ ID NO: 1.

In a preferred embodiment the present invention relates to the antisense oligonucleotide, wherein the contiguous nucleotide sequence is at least 90% complementary, such as fully complementary, to an intron region present in the pre-mRNA of human FNDC3B, selected from position 145-72824 on SEQ ID NO: 1), position 72964-93844 on SEQ ID NO: 1, and position 93920-147084 on SEQ ID NO: 1.

In another preferred embodiment the present invention relates to the antisense oligonucleotide, wherein the contiguous nucleotide sequence is at least 90% complementary, such as fully complementary, to position 145-72824 of on the human pre-mRNA of FNDC3B encoding target nucleic acid, such as SEQ ID NO: 1.

In another preferred embodiment the present invention relates to the antisense oligonucleotide, wherein the contiguous nucleotide sequence is at least 90% complementary, such as fully complementary, to SEQ ID NO 9.

In another preferred embodiment the present invention relates to the antisense oligonucleotide, wherein the contiguous nucleotide sequence is at least 90% complementary, such as fully complementary, to SEQ ID NO 10.

In another preferred embodiment the present invention relates to the antisense oligonucleotide, wherein the contiguous nucleotide sequence is at least 90% complementary, such as fully complementary, to SEQ ID NO 19.

In another preferred embodiment the present invention relates to the antisense oligonucleotide, wherein the contiguous nucleotide sequence is at least 90% complementary, such as fully complementary, to SEQ ID NO 20.

In some embodiment the oligonucleotide or contiguous nucleotide sequence is complementary to a region of the target nucleic acid, wherein the target nucleic acid region is selected from the group consisting of position 54366-54385, 54407-54426, 54448-54467, 54489-54508, 54530-54549, 54571-54590, 54612-54631; 54367-54384, 54408-54425, 54449-54466, 54490-54507, 54531-54548, 54572-54589, 54613-54630; 54368-54383, 54409-54424, 54450-54465, 54491-54506, 54532-54547, 54573-54588, 54614-54629; 54366-54379, 54407-54420, 54448-54461, 54489-54502, 54530-54543, 54571-54584, 54612-54625, 351016-351033; 351016-351029; 351016-351035; 351 01 8-351 33 of SEQ ID NO: 1.

According to one aspect of the invention, the target sequence is repeated within the target nucleic acid, i.e. at least two identical target nucleotide sequences (target regions) of at least 10 nucleotides in length occur in the target nucleic acid at different positions. A repeated target region is generally between 10 and 50 nucleotides, such as between 11 and 30 nucleotides, such as between 12 and 25 nucleotides, such as between 13 and 22 nucleotides, such as between 14 and 20 nucleotides, such as between 15 and 19 nucleotides, such as between 16 and 18 nucleotides. In a preferred embodiment the repeated target region is between 14 and 20 nucleotides.

In one aspect the invention provides antisense oligonucleotides wherein the contiguous nucleotide sequence is at least 90% complementary, such as fully complementary, to a target region that is repeated at least 2 times across the target nucleic acid of SEQ ID NO: 1. The effect of this is that several oligonucleotide compounds (with the same sequence) can hybridize to one or more target regions on the same target nucleic acid (at the same time), which may result in multiple cleavage events of the target nucleic acid when the oligonucleotide is administered to a cell or an animal or a human.

In some embodiments the oligonucleotide or the contiguous nucleotide sequence is at least 90% complementary, such as fully complementary to a target region that is repeated at least at least 3 repeated target regions, such as at least 4, 5, 6, 7, 8, 9 or 10 repeated target regions, or more than 10 repeated target regions.

In a further embodiment the antisense oligonucleotide comprises a contiguous nucleotide sequence that is at least 90% complementary, such as fully complementary, to a target region of 10-22, such as 14-20, nucleotides in length of the target nucleic acid of SEQ ID NO: 1, wherein the target region is repeated at least 2 or more times across the intons of the target nucleic acid.

In some embodiments, the antisense oligonucleotide of the invention or the contiguous nucleotide sequence thereof is complementary to at least 6 repeated target regions in SEQ ID NO: 10.

In some embodiments, the oligonucleotide of the invention comprises or consists of 10 to 35 nucleotides in length, such as from 10 to 30, such as 11 to 22, such as from 12 to 20, such as from 14 to 18 or 14 to 16 contiguous nucleotides in length. Advantageously, the oligonucleotide comprises or consists of 14 to 20 nucleotides in length.

It is to be understood that any range given herein includes the range endpoints.

In some embodiments, the oligonucleotide or contiguous nucleotide sequence thereof comprises or consists of 22 or less nucleotides, such as 20 or less nucleotides, such as 18 or less nucleotides, such as less than 18, such as 14, 15, 16 or 17 nucleotides.

In some embodiments, the antisense oligonucleotide or contiguous nucleotide sequence thereof, comprises or consists of 10 to 30 nucleotides in length with at least 90% identity, preferably 100% identity, to a sequence selected from the group consisting of SEQ ID NO: 11 to 18.

In some embodiments, the antisense oligonucleotide or contiguous nucleotide sequence thereof comprises or consists of 10 to 30 contiguous nucleotides in length with at least 90% identity, preferably 100% identity, to a sequence selected from SEQ ID NO: 15 to 18.

In some embodiments, the antisense oligonucleotide or contiguous nucleotide sequence thereof comprises or consists of 10 to 30 contiguous nucleotides in length with at least 90% identity, preferably 100% identity, to a sequence selected from SEQ ID NO: 11 to 14.

In some embodiments, the antisense oligonucleotide or contiguous nucleotide sequence thereof comprises or consists of 12 to 20 contiguous nucleotides in length with at least 90% identity, preferably 100% identity, to a sequence selected from SEQ ID NO: 15 to 18.

In some embodiments, the antisense oligonucleotide or contiguous nucleotide sequence thereof comprises or consists of 12 to 20 contiguous nucleotides in length with at least 90% identity, preferably 100% identity, to a sequence selected from SEQ ID NO: 11 to 14.

In some embodiments, the antisense oligonucleotide or contiguous nucleotide sequence thereof comprises or consists of 14 to 20 contiguous nucleotides in length with at least 90% identity, preferably 100% identity, to a sequence selected from SEQ ID NO: 15 to 18.

In some embodiments, the antisense oligonucleotide or contiguous nucleotide sequence thereof comprises or consists of 14 to 20 contiguous nucleotides in length with at least 90% identity, preferably 100% identity, to a sequence selected from SEQ ID NO: 11 to 14.

In some embodiments, the antisense oligonucleotide or contiguous nucleotide sequence (motif sequence) thereof consists of a sequence selected from SEQ ID NO: 15 to 18.

In some embodiments, the antisense oligonucleotide or contiguous nucleotide sequence (motif sequence) thereof consists of a sequence selected from SEQ ID NO: 11 to 14.

It is understood that the contiguous nucleobase sequences (motif sequence) can be modified to for example increase nuclease resistance and/or binding affinity to the target nucleic acid. Modifications are described in the definitions and in the following paragraphs. Table 4 lists preferred designs of each motif sequence.

The pattern in which the modified nucleosides (such as high affinity modified nucleosides) are incorporated into the oligonucleotide sequence is generally termed oligonucleotide design.

The oligonucleotides of the invention are designed with modified nucleosides and DNA nucleosides. Advantageously, high affinity modified nucleosides are used.

In an embodiment, the oligonucleotide comprises at least 1 modified nucleoside, such as at least 2, at least 3, at least 4, at least 5, at least 6, at least 7, at least 8, at least 9, at least 10, at least 11, at least 12, at least 13, at least 14, at least 15 or at least 16 modified nucleosides. In an embodiment the oligonucleotide comprises from 1 to 10 modified nucleosides, such as from 2 to 9 modified nucleosides, such as from 3 to 8 modified nucleosides, such as from 4 to 7 modified nucleosides, such as 6 or 7 modified nucleosides. Suitable modifications are described in the “Definitions” section under “modified nucleoside”, “high affinity modified nucleosides”, “sugar modifications”, “2′ sugar modifications” and Locked nucleic acids (LNA)”.

In an embodiment, the oligonucleotide comprises one or more sugar modified nucleosides, such as 2′ sugar modified nucleosides. Preferably the oligonucleotide of the invention comprise one or more 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides. It is advantageous if one or more of the modified nucleoside(s) is a locked nucleic acid (LNA).

In a further embodiment the oligonucleotide comprises at least one modified internucleoside linkage. Suitable internucleoside modifications are described in the “Definitions” section under “Modified internucleoside linkage”. It is advantageous if at least 75%, such as all, the internucleoside linkages within the contiguous nucleotide sequence are phosphorothioate or boranophosphate internucleoside linkages. In some embodiments all the internucleotide linkages in the contiguous sequence of the oligonucleotide are phosphorothioate linkages.

In some embodiments, the oligonucleotide of the invention comprises at least one LNA nucleoside, such as 1, 2, 3, 4, 5, 6, 7, or 8 LNA nucleosides, such as from 2 to 6 LNA nucleosides, such as from 3 to 7 LNA nucleosides, 4 to 8 LNA nucleosides or 3, 4, 5, 6, 7 or 8 LNA nucleosides. In some embodiments, at least 75% of the modified nucleosides in the oligonucleotide are LNA nucleosides, such as 80%, such as 85%, such as 90% of the modified nucleosides are LNA nucleosides. In a still further embodiment all the modified nucleosides in the oligonucleotide are LNA nucleosides. In a further embodiment, the oligonucleotide may comprise both beta-D-oxy-LNA, and one or more of the following LNA nucleosides: thio-LNA, amino-LNA, oxy-LNA, ScET and/or ENA in either the beta-D or alpha-L configurations or combinations thereof. In a further embodiment, all LNA cytosine units are 5-methyl-cytosine. It is advantageous for the nuclease stability of the oligonucleotide or contiguous nucleotide sequence to have at least 1 LNA nucleoside at the 5′ end and at least 2 LNA nucleosides at the 3′ end of the nucleotide sequence.

In an embodiment of the invention the oligonucleotide of the invention is capable of recruiting RNase H.

In the current invention an advantageous structural design is a gapmer design as described in the “Definitions” section under for example “Gapmer”, “LNA Gapmer”, “MOE gapmer” and “Mixed Wing Gapmer” “Alternating Flank Gapmer”. The gapmer design includes gapmers with uniform flanks, mixed wing flanks, alternating flanks, and gapbreaker designs. In the present invention it is advantageous if the oligonucleotide of the invention is a gapmer with an F-G-F′ design. In some embodiments the gapmer is an LNA gapmer with uniform flanks.

In some embodiments of the invention the LNA gapmer is selected from the following uniform flank designs 3-12-3, 3-9-2, 4-10-2, or 4-9-3.

Exemplary Compounds of the Invention

In the exemplified oligonucleotide compounds, capital letters represent beta-D-oxy LNA nucleosides, lowercase letters represent DNA nucleosides, all LNA C are 5-methyl cytosine, and 5-methyl DNA cytosines may be present in the gap region and would be presented by “e” or ^{m}c, all internucleoside linkages are phosphorothioate internucleoside linkages.

For some embodiments of the invention, the oligonucleotide is selected from the group of oligonucleotide compounds with CMP-ID-NO: 11_1; 12_1; 13_1 and 14_1.

In one embodiment of the invention the oligonucleotide is CMP-ID-NO: 13_1 or 14_1.

For some embodiments of the invention, the oligonucleotide is selected from the group of oligonucleotide compounds with CMP-ID-NO 15_1; 16_1; 17_1; 18_1.

Method of Manufacture

In a further aspect, the invention provides methods for manufacturing the oligonucleotides of the invention comprising reacting nucleotide units and thereby forming covalently linked contiguous nucleotide units comprised in the oligonucleotide. Preferably, the method uses phophoramidite chemistry (see for example Caruthers et al, 1987, Methods in Enzymology vol. 154, pages 287-313). In a further embodiment the method further comprises reacting the contiguous nucleotide sequence with a conjugating moiety (ligand). In a further aspect a method is provided for manufacturing the composition of the invention, comprising mixing the oligonucleotide or conjugated oligonucleotide of the invention with a pharmaceutically acceptable diluent, solvent, carrier, salt and/or adjuvant.

Pharmaceutical Salt

In a further aspect the invention provides a pharmaceutically acceptable salt of the antisense oligonucleotide or a conjugate thereof. In a preferred embodiment, the pharmaceutically acceptable salt is a sodium or a potassium salt.

Pharmaceutical Composition

In a further aspect, the invention provides pharmaceutical compositions comprising any of the aforementioned oligonucleotides and/or oligonucleotide conjugates or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to, sodium and potassium salts. In some embodiments the pharmaceutically acceptable diluent is sterile phosphate buffered saline. In some embodiments the oligonucleotide is used in the pharmaceutically acceptable diluent at a concentration of 50-300 μM solution.

Suitable formulations for use in the present invention are found in Remington's Pharmaceutical Sciences, Mack Publishing Company, Philadelphia, Pa., 17th ed., 1985. For a brief review of methods for drug delivery, see, e.g., Langer (Science 249:1527-1533, 1990). WO 2007/031091 provides further suitable and preferred examples of pharmaceutically acceptable diluents, carriers and adjuvants (hereby incorporated by reference). Suitable dosages, formulations, administration routes, compositions, dosage forms, combinations with other therapeutic agents, pro-drug formulations are also provided in WO2007/031091.

Oligonucleotides or oligonucleotide conjugates of the invention may be mixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

These compositions may be sterilized by conventional sterilization techniques, or may be sterile filtered. The resulting aqueous solutions may be packaged for use as is, or lyophilized, the lyophilized preparation being combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

In some embodiments, the oligonucleotide or oligonucleotide conjugate of the invention is a prodrug. In particular with respect to oligonucleotide conjugates the conjugate moiety is cleaved of the oligonucleotide once the prodrug is delivered to the site of action, e.g. the target cell.

Applications

The oligonucleotides of the invention may be utilized as research reagents for, for example, diagnostics, therapeutics and prophylaxis.

In research, such oligonucleotides may be used to specifically modulate the synthesis of FNDC3B protein in cells (e.g. in vitro cell cultures) and experimental animals thereby facilitating functional analysis of the target or an appraisal of its usefulness as a target for therapeutic intervention. Typically the target modulation is achieved by degrading or inhibiting the mRNA producing the protein, thereby prevent protein formation or by degrading or inhibiting a modulator of the gene or mRNA producing the protein. Further advantages may be achieved by targeting pre-mRNA thereby preventing formation of the mature mRNA.

If employing the oligonucleotide of the invention in research or diagnostics the target nucleic acid may be a cDNA or a synthetic nucleic acid derived from DNA or RNA.

The present invention provides an in vivo or in vitro method for modulating FNDC3B expression in a target cell which is expressing FNDC3B, said method comprising administering an oligonucleotide of the invention in an effective amount to said cell.

In some embodiments, the target cell, is a mammalian cell in particular a human cell. The target cell may be an in vitro cell culture or an in vivo cell forming part of a tissue in a mammal. In preferred embodiments the target cell is present in, or is isolate or derived from, stomach, gut, liver, lungs, pancreas, islet of Langerhans, placenta, or testis.

In diagnostics the oligonucleotides may be used to detect and quantitate FNDC3B expression in cell and tissues by northern blotting, in-situ hybridisation or similar techniques.

For therapeutics, an animal or a human, suspected of having a disease or disorder, which can be treated by reducing the expression of FNDC3B.

The invention provides methods for treating or preventing a disease, comprising administering a therapeutically or prophylactically effective amount of an antisense oligonucleotide, an oligonucleotide conjugate, a pharmaceutical salt or a pharmaceutical composition of the invention to a subject suffering from or susceptible to the disease.

The invention also relates to an antisense oligonucleotide, a pharmaceutical salt, a composition or a conjugate as defined herein for use as a medicament.

The antisense oligonucleotide, oligonucleotide conjugate or a pharmaceutical composition according to the invention is typically administered in an effective amount.

The invention also provides for the use of the antisense oligonucleotide or oligonucleotide conjugate of the invention as described for the manufacture of a medicament for the treatment of a disorder as referred to herein, or for a method of the treatment of as a disorder as referred to herein.

The disease or disorder, as referred to herein, is associated with the increased expression of FNDC3B.

The methods of the invention are preferably employed for treatment or prophylaxis against diseases caused by abnormally high levels and/or activity of FNDC3B.

The invention further relates to use of an antisense oligonucleotide, oligonucleotide conjugate, a pharmaceutical salt or a pharmaceutical composition as defined herein for the manufacture of a medicament for the treatment of abnormally high levels and/or activity of FNDC3B.

In one embodiment, the invention relates to oligonucleotides, oligonucleotide conjugates or pharmaceutical compositions for use in the treatment of diseases or disorders selected from cancers, such as hepatocellular carcinoma and/or acute myeloid leukemia.

Administration

In a non-limiting embodiment the antisense oligonucleotide, a conjugate, a pharmaceutical salt or pharmaceutical compositions of the present invention are administered by a parenteral route including intravenous, intraarterial, subcutaneous, intraperitoneal or intramuscular injection or infusion. In one non-limiting embodiment the active oligonucleotide or oligonucleotide conjugate is administered intravenously.

In some embodiments, the antisense oligonucleotide, oligonucleotide conjugate or pharmaceutical composition of the invention is administered at a dose of 0.1-15 mg/kg, such as from 0.2-10 mg/kg, such as from 0.25-5 mg/kg. The administration can be once a week, every 2^{nd} week, every third week or even once a month.

The invention also provides for the use of the oligonucleotide or oligonucleotide conjugate of the invention as described for the manufacture of a medicament wherein the medicament is in a dosage form for intravenous administration.

Combination Therapies

In some embodiments the antisense oligonucleotide, oligonucleotide conjugate or pharmaceutical composition of the invention is for use in a combination treatment with another therapeutic agent. The therapeutic agent can for example be the standard of care for the diseases or disorders described above.

### Embodiments

The following embodiments of the present invention may be used in combination with any other embodiments described herein.

1. An antisense oligonucleotide of 10 to 50 nucleotides in length, which comprises a contiguous nucleotide sequence of 10 to 30 nucleotides in length with at least 90% complementarity to a mammalian FNDC3B encoding target nucleic acid, wherein the antisense oligonucleotide is capable of reducing the expression of the mammalian FNDC3B encoding target nucleic acid, in a cell.

2. The antisense oligonucleotide according to embodiment 1, wherein the contiguous nucleotide sequence is at least 90% complementary to a sequence selected from the group consisting of SEQ ID NO 1, 2, 3, 4, 5, 6, 7 and 8, or a naturally occurring variant thereof.

3. The antisense oligonucleotide of embodiment 1 or 2, wherein the contiguous nucleotide sequence is fully complementary to the mammalian FNDC3B encoding target nucleic acid.

4. The antisense oligonucleotide according to any one of embodiments 1 to 3, wherein the contiguous nucleotide sequence is at least 90% complementary, such as fully complementary, to an intron region present in the pre-mRNA of mammalian FNDC3B target nucleic acid (e.g. SEQ ID NO 1).

5. The antisense oligonucleotide according to any one of embodiments 1-4, wherein the contiguous nucleotide sequence is at least 90% complementary, such as fully complementary, to an intron region present in the pre-mRNA of human FNDC3B, selected from position 145-72824 on SEQ ID NO: 1, position 72964-93844 on SEQ ID NO: 1, and position 93920-147084 on SEQ ID NO: 1.

6. The antisense oligonucleotide according to any one of embodiments 1-5, wherein the contiguous nucleotide sequence is at least 90% complementary, such as fully complementary, to a position 145-72824 on the human pre-mRNA of mammalian FNDC3B encoding target nucleic acid, such as SEQ ID NO: 1.

7. The antisense oligonucleotide according to any one of embodiments 1-6, wherein the contiguous nucleotide sequence is at least 90% complementary, such as fully complementary, to SEQ ID NO 9.

8. The antisense oligonucleotide according to any one of embodiments 1-7, wherein the contiguous nucleotide sequence is at least 90% complementary, such as fully complementary, to a target region of SEQ ID NO 1, selected from the group consisting of position 54366-54385, 54407-54426, 54448-54467, 54489-54508, 54530-54549, 54571-54590, 54612-54631; 54367-54384, 54408-54425, 54449-54466, 54490-54507, 54531-54548, 54572-54589, 54613-54630; 54368-54383, 54409-54424, 54450-54465, 54491-54506, 54532-54547, 54573-54588, 54614-54629; 54366-54379, 54407-54420, 54448-54461, 54489-54502, 54530-54543, 54571-54584, 54612-54625; 351016-351033; 351016-351029; 351016-351035; 351 01 8-351 33 of SEQ ID NO:

9. The antisense oligonucleotide according to any one of embodiments 1-8, wherein the contiguous nucleotide sequence is at least 90% complementary, such as fully complementary, to more than one target region of SEQ ID NO 1, said one or more target regions being identical to each other.

10. The antisense oligonucleotide of embodiment 1 to 3, wherein the oligonucleotide is capable of hybridizing to a target nucleic acid selected from the group consisting of SEQ ID NO: 1 to 8 with a ΔG° below-10 kcal.

11. The antisense oligonucleotide of embodiment 9 or 10, wherein the target nucleic acid is

RNA.

12. The antisense oligonucleotide of embodiment 11, wherein the RNA is mRNA.

13. The antisense oligonucleotide of embodiment 12, wherein the mRNA is pre-RNA or mature RNA.

14. The antisense oligonucleotide of any one of embodiments 1-13, wherein the contiguous nucleotide sequence comprises or consists of at least 10 contiguous nucleotides, particularly 11, 12, 13, 14, 15, 16, 17, 18, 19, 20, 21, 22, 23, 24, 25, 26, 27, 28, or 29 contiguous nucleotides.

15. The antisense oligonucleotide of any one of embodiments 1-13, wherein the contiguous nucleotide sequence comprises or consists of from 12 to 22 nucleotides.

16. The antisense oligonucleotide of any one of embodiments 1 to 15, wherein the contiguous nucleotide sequence comprises or consists of from 12-18 nucleotides.

17. The antisense oligonucleotide of any one of embodiments 1 to -16, wherein the antisense oligonucleotide comprises or consists of 10 to 35 nucleotides in length.

18. The antisense oligonucleotide of any one of embodiments 1 to 17, wherein the antisense oligonucleotide comprises or consists of 11 to 22 nucleotides in length.

19. The antisense oligonucleotide of any one of embodiments 17 to 19, wherein the oligonucleotide comprises or consists of 12 to 18 nucleotides in length.

20. The antisense oligonucleotide of any one of embodiments 1-19, wherein the oligonucleotide or contiguous nucleotide sequence is single stranded.

21. The antisense oligonucleotide of any one of embodiments 1-20 wherein the oligonucleotide is not siRNA nor self-complementary.

22. The antisense oligonucleotide of embodiment 1-21, wherein the contiguous nucleotide sequence comprises or consists of a sequence selected from SEQ ID NO: 11, 12, 13, 14, 15, 16, 17 and 18.

23. The antisense oligonucleotide of any one of embodiments 1-22, wherein the contiguous nucleotide sequence has zero to three mismatches compared to the target nucleic acid it is complementary to.

24. The antisense oligonucleotide of embodiment 23, wherein the contiguous nucleotide sequence has one mismatch compared to the target nucleic acid.

25. The antisense oligonucleotide of embodiment 23, wherein the contiguous nucleotide sequence has two mismatches compared to the target nucleic acid.

26. The antisense oligonucleotide of embodiment 23, wherein the contiguous nucleotide sequence is fully complementary to the target nucleic acid sequence.

27. The antisense oligonucleotide of embodiment 1-26, comprising one or more modified nucleosides.

28. The antisense oligonucleotide of embodiment 27, wherein the one or more modified nucleoside is a high-affinity modified nucleosides.

29. The antisense oligonucleotide of embodiment 27 or 28, wherein the one or more modified nucleoside is a 2′ sugar modified nucleoside.

30. The antisense oligonucleotide of embodiment 29, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, 2′-fluoro-ANA and LNA nucleosides.

31. The antisense oligonucleotide of embodiments 27 to 30, wherein the one or more modified nucleoside is a LNA nucleoside.

32. The antisense oligonucleotide of embodiment 31, wherein the modified LNA nucleoside is oxy-LNA.

33. The antisense oligonucleotide of embodiment 32, wherein the modified nucleoside is beta-D-oxy-LNA.

34. The antisense oligonucleotide of embodiment 31, wherein the modified nucleoside is thio-LNA.

35. The antisense oligonucleotide of embodiment 31, wherein the modified nucleoside is amino-LNA.

36. The antisense oligonucleotide of embodiment 31, wherein the modified nucleoside is cET.

37. The antisense oligonucleotide of embodiment 31, wherein the modified nucleoside is ENA.

38. The antisense oligonucleotide of embodiment 31, wherein the modified LNA nucleoside is selected from beta-D-oxy-LNA, alpha-L-oxy-LNA, beta-D-amino-LNA, alpha-L-amino-LNA, beta-D-thio-LNA, alpha-L-thio-LNA, (S)cET, (R)cET beta-D-ENA and alpha-L-ENA.

39. The antisense oligonucleotide of any one of embodiments 1-38, wherein the oligonucleotide comprises at least one modified internucleoside linkage.

40. The antisense oligonucleotide of embodiment 39, wherein the modified internucleoside linkage is nuclease resistant.

41. The antisense oligonucleotide of embodiment 39 or 40, wherein at least 50% of the internucleoside linkages within the contiguous nucleotide sequence are phosphorothioate internucleoside linkages or boranophosphate internucleoside linkages.

42. The antisense oligonucleotide of embodiment 39 or 40, wherein all the internucleoside linkages within the contiguous nucleotide sequence are phosphorothioate internucleoside linkages.

43. The antisense oligonucleotide of embodiment 1-42, wherein the antisense oligonucleotide is capable of recruiting RNase H.

44. The antisense oligonucleotide of embodiment 43, wherein the antisense oligonucleotide or contiguous nucleotide sequence thereof comprises a gapmer.

45. The antisense oligonucleotide of embodiment 43 or 44, wherein the antisense oligonucleotide or contiguous nucleotide sequence thereof consists of or comprises a gapmer of formula 5′-F-G-F′-3′, where region F and F′ independently comprise or consist of 1-7 modified nucleosides and G is a region between 6 and 17 nucleosides which are capable of recruiting RNaseH, such as a region comprising 6 to 17 DNA nucleosides

46. The antisense oligonucleotide of embodiment 45, wherein the modified nucleoside is a 2′ sugar modified nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

47. The antisense oligonucleotide of embodiment 45 or 46, wherein one or more of the modified nucleosides in region F and F′ is a LNA nucleoside.

48. The antisense oligonucleotide of embodiment 47, wherein all the modified nucleosides in region F and F′ are LNA nucleosides.

49. The antisense oligonucleotide of embodiment 48, wherein region F and F′ consist of LNA nucleosides.

50. The antisense oligonucleotide of embodiment 47-49, wherein all the modified nucleosides in region F and F′ are oxy-LNA nucleosides.

51. The antisense oligonucleotide of embodiment 47, wherein at least one of region F or F′ further comprises at least one 2′ substituted modified nucleoside independently selected from the group consisting of 2′-O-alkyl-RNA, 2′-O-methyl-RNA, 2′-alkoxy-RNA, 2′-O-methoxyethyl-RNA, 2′-amino-DNA and 2′-fluoro-DNA.

52. The antisense oligonucleotide of embodiment 47-51, wherein the RNaseH recruiting nucleosides in region G are independently selected from DNA, alpha-L-LNA, C4′ alkylated DNA, ANA and 2′F-ANA and UNA.

53. The antisense oligonucleotide of embodiment 52, wherein the nucleosides in region G is

DNA and/or alpha-L-LNA nucleosides.

54. The antisense oligonucleotide of embodiment 52 or 53, wherein region G consists of at least 75% DNA nucleosides.

55. The antisense oligonucleotide according to any one of embodiments 1 to 54, wherein wherein the antisense oligonucleotide, or contiguous nucleotide sequence thereof is selected from the group consisting of GaatttgctagtggtggtGG; GCTagtggtggtGG; AATttgctagtggtgGTG; ATTTgctagtggtgGT; TTatttctacagtttccAGA; ATTTctacagtttccAGA; CTacagtttcCAGA; and ATTTctacagtttCCA; wherein capital letters represent LNA nucleosides, such as beta-D-oxy LNA nucleosides, lower case letters represent DNA nucleosides, opionally, all LNA C are 5-methyl cytosine, and all internucleoside linkages are phosphorothioate internucleoside linkages.

56. The oligonucleotide of embodiment 55, wherein the oligonucleotide is selected from CMP ID NO:11_1; 12_1; 13_1; 14_1; 15_1; 16_1; 17_1 and 18_1.

57. A conjugate comprising the antisense oligonucleotide according to any one of claims 1-56, and at least one conjugate moiety covalently attached to said oligonucleotide.

58. The antisense oligonucleotide conjugate of embodiment 57, wherein the conjugate moiety is selected from carbohydrates, cell surface receptor ligands, drug substances, hormones, lipophilic substances, polymers, proteins, peptides, toxins, vitamins, viral proteins or combinations thereof.

59. The antisense oligonucleotide conjugate of embodiment 57 or 58, wherein the conjugate moiety is capable of binding to the asialoglycoprotein receptor.

60. The antisense oligonucleotide conjugate of any one of embodiments 57-59, comprising a linker which is positioned between the antisense oligonucleotide and the conjugate moiety.

61. The antisense oligonucleotide conjugate of embodiment 60, wherein the linker is a physiologically labile linker.

62. The antisense oligonucleotide conjugate of embodiment 61, wherein the physiologically labile linker is nuclease susceptible linker.

63. The antisense oligonucleotide conjugate of embodiment 61 or 62, wherein the oligonucleotide has the formula D′-F-G-F′ or F-G-F′-D″, wherein F, F′ and G are as defined in embodiments 47-56 and D′ or D″ comprises 1, 2 or 3 DNA nucleosides with phosphorothioate internucleoside linkages.

64. A pharmaceutically acceptable salt of the antisense oligonucleotide according to any one of embodiments 1-56 or the conjugate according to any of embodiments 57 to 63.

65. A pharmaceutical composition comprising the antisense oligonucleotide of embodiment 1-56 or a conjugate of embodiment 57-63 and a pharmaceutically acceptable diluent, carrier, salt and/or adjuvant.

66. A method for manufacturing the antisense oligonucleotide of any one of embodiments 1-56, comprising reacting nucleotide units thereby forming covalently linked contiguous nucleotide units comprised in the oligonucleotide.

67. The method of embodiment 66, further comprising reacting the contiguous nucleotide sequence with a non-nucleotide conjugation moiety.

68. A method for manufacturing the composition of embodiment 65, comprising mixing the oligonucleotide with a pharmaceutically acceptable diluent, carrier, salt and/or adjuvant.

69. An in vivo or in vitro method for reducing FNDC3B expression in a target cell which is expressing the mammalianFNDC3B, said method comprising administering an antisense oligonucleotide of embodiments 1-56 or a conjugate of embodiments 57-63 or the pharmaceutically acceptable salt of embodiment 64 or the pharmaceutical composition of embodiment 65 in an effective amount to said cell.

70. A method for treating, alleviating or preventing a disease comprising administering a therapeutically or prophylactically effective amount of an antisense oligonucleotide of embodiments 1-56 or a conjugate of embodiments 57-63 or the pharmaceutically acceptable salt of embodiment 64 or the pharmaceutical composition of embodiment 65 to a subject suffering from or susceptible to the disease.

71. The antisense oligonucleotide of embodiments 1-56 or a conjugate of embodiments 57-63 or the pharmaceutically acceptable salt of embodiment 64 or the pharmaceutical composition of embodiment 65, for use as a medicament for treatment, alleviation or prevention of a disease in a subject.

72. Use of the antisense oligonucleotide of embodiments 1-56 or a conjugate of embodiments 57-63 or the pharmaceutically acceptable salt of embodiment 64 for the preparation of a medicament for treatment, alleviation or prevention of a disease in a subject.

73. The method, the antisense oligonucleotide or the use of embodiments 70-72, wherein the disease is associated with in vivo activity of FNDC3B.

74. The method, the antisense oligonucleotide or the use of embodiments 70-73, wherein the disease is associated with overexpression of FNDC3B gene and/or abnormal levels of FNDC3B protein.

75. The method, the antisense oligonucleotide or the use of embodiment 74, wherein the FNDC3B gene expression is reduced by at least 30%, or at least or at least 40%, or at least 50%, or at least 60%, or at least 70%, or at least 80%, or at least 90%, or at least 95% compared to the expression without the oligonucleotide of embodiments 1-56 or a conjugate of embodiment 57-63 or the pharmaceutically acceptable salt of embodiment 64 or the pharmaceutical composition of embodiment 65.

76. The method, the antisense oligonucleotide or the use of embodiments 70-74, wherein the disease is cancer, selected from acute myeloid leukemia and hepatocellular carcinoma.

77. The method, the antisense oligonucleotide or the use of embodiments 70-76, wherein the subject is a mammal.

78. The method, the antisense oligonucleotide or the use of embodiment 77, wherein the mammal is human.

### Examples

Materials and methods

TABLE 4
list of oligonucleotide motif sequences (indicated by SEQ ID NO), designs of these,
as well as specific oligonucleotide compounds (indicated by CMP ID NO) designed
based on the motif sequence.

| SEQ | | | | CMP | |
|---|---|---|---|---|---|
| ID | | | Oligonucleotide | ID | Start position |
| NO | Motif sequence | Design | Compound | NO | on SEQ ID NO: 1* |
| 11 | GAATTTGCTAGTGGTGGTGG | 1-17-2 | GaatttgctagtggtggtGG | 11_1 | 54366, 54407, |
| | | | | | 54448, 54489, |
| | | | | | 54530, 54571, |
| | | | | | 54612 |
| 12 | GCTAGTGGTGGTGG | 3-9-2 | GCTagtggtggtGG | 12_1 | 54366, 54407, |
| | | | | | 54448, 54489, |
| | | | | | 54530, 54571, |
| | | | | | 54612 |
| 13 | AATTTGCTAGTGGTGGTG | 3-12-3 | AATttgctagtggtgGTG | 13_1 | 54367, 54408, |
| | | | | | 54449, 54490, |
| | | | | | 54531, 54572, |
| | | | | | 54613 |
| 14 | ATTTGCTAGTGGTGGT | 4-10-2 | ATTTgctagtggtgGT | 14_1 | 54368, 54409, |
| | | | | | 54450, 54491, |
| | | | | | 54532, 54573, |
| | | | | | 54614 |
| 15 | TTATTTCTACAGTTTCCAGA | 2-15-3 | TTatttctacagtttccAGA | 15_1 | 351016 |
| 16 | ATTTCTACAGTTTCCAGA | 4-11-3 | ATTTctacagtttccAGA | 16_1 | 351016 |
| 17 | CTACAGTTTCCAGA | 2-8-4 | CTacagtttcCAGA | 17_1 | 351016 |
| 18 | ATTTCTACAGTTTCCA | 4-9-3 | ATTTctacagtttCCA | 18_1 | 351018 |

*multiple numbers refers to repeat targeting compounds

Motif sequences represent the contiguous sequence of nucleobases present in the oligonucleotide.

Designs refer to the gapmer design, F-G-F′, where each number represents the number of consecutive modified nucleosides, e.g. 2′ sugar modified nucleosides (first number=5′ flank), followed by the number of DNA nucleosides (second number=gap region), followed by the number of modified nucleosides, e.g. 2′ sugar modified nucleosides (third number=3′ flank), optionally preceded by or followed by further repeated regions of DNA and LNA, which are not necessarily part of the contiguous sequence that is complementary to the target nucleic acid.

Oligonucleotide compounds represent specific designs of a motif sequence. Capital letters represent beta-D-oxy LNA nucleosides, lowercase letters represent DNA nucleosides, all LNA C are 5-methyl cytosine, and 5-methyl DNA cytosines are presented by “e”, all internucleoside linkages are phosphorothioate internucleoside linkages.

Oligonucleotide Synthesis

Oligonucleotide synthesis is generally known in the art. Below is a protocol which may be applied. The oligonucleotides of the present invention may have been produced by slightly varying methods in terms of apparatus, support and concentrations used.

Oligonucleotides are synthesized on uridine universal supports using the phosphoramidite approach on an Oligomaker 48 at 1 μmol scale. At the end of the synthesis, the oligonucleotides are cleaved from the solid support using aqueous ammonia for 5-16 hours at 60° C. The oligonucleotides are purified by reverse phase HPLC (RP-HPLC) or by solid phase extractions and characterized by UPLC, and the molecular mass is further confirmed by ESI-MS.

Elongation of the Oligonucleotide:

The coupling of β-cyanoethyl-phosphoramidites (DNA-A(Bz), DNA-G(ibu), DNA-C(Bz), DNA-T, LNA-5-methyl-C(Bz), LNA-A(Bz), LNA-G(dmf), or LNA-T) is performed by using a solution of 0.1 M of the 5′-O-DMT-protected amidite in acetonitrile and DCI (4,5-dicyanoimidazole) in acetonitrile (0.25 M) as activator. For the final cycle a phosphoramidite with desired modifications can be used, e.g. a C6 linker for attaching a conjugate group or a conjugate group as such. Thiolation for introduction of phosphorthioate linkages is carried out by using xanthane hydride (0.01 M in acetonitrile/pyridine 9:1). Phosphordiester linkages can be introduced using 0.02 M iodine in THF/Pyridine/water 7:2:1. The rest of the reagents are the ones typically used for oligonucleotide synthesis.

For post solid phase synthesis conjugation a commercially available C6 aminolinker phorphoramidite can be used in the last cycle of the solid phase synthesis and after deprotection and cleavage from the solid support the aminolinked deprotected oligonucleotide is isolated. The conjugates are introduced via activation of the functional group using standard synthesis methods.

Purification by RP-HPLC:

The crude compounds are purified by preparative RP-HPLC on a Phenomenex Jupiter C18 10μ150×10 mm column. 0.1 M ammonium acetate pH 8 and acetonitrile is used as buffers at a flow rate of 5 mL/min. The collected fractions are lyophilized to give the purified compound typically as a white solid.

### Abbreviations

DCI: 4,5-Dicyanoimidazole

DCM: Dichloromethane

DMF: Dimethylformamide

DMT: 4,4′-Dimethoxytrityl

THF: Tetrahydrofurane

Bz: Benzoyl

Ibu: Isobutyryl

RP-HPLC: Reverse phase high performance liquid chromatography

Tₘ Assay:

Oligonucleotide and RNA target (phosphate linked, PO) duplexes are diluted to 3 mM in 500 ml RNase-free water and mixed with 500 ml 2×Tₘ-buffer (200 mM NaCl, 0.2 mM EDTA, 20 mM Naphosphate, pH 7.0). The solution is heated to 95° C. for 3 min and then allowed to anneal in room temperature for 30 min. The duplex melting temperatures (Tₘ) is measured on a Lambda 40 UV/VIS Spectrophotometer equipped with a Peltier temperature programmer PTP6 using PE Templab software (Perkin Elmer). The temperature is ramped up from 20° C. to 95° C. and then down to 25° C., recording absorption at 260 nm. First derivative and the local maximums of both the melting and annealing are used to assess the duplex Tₘ.

### Example 1—Testing In Vitro Efficacy and Potency

Oligonucleotides targeting one region as well as oligonucleotides targeting at least two target regions being identical to each other on FNDC3B (SEQ ID NO: 1) were tested in an in vitro experiment in HeLa cells. EC50 (potency) and max kd (efficacy) was assessed for the single targeting and repeat targeting oligonucleotides.

Cell Lines

The HeLa cell line was purchased from European Collection of Authenticated Cell Cultures (ECACC) and maintained as recommended by the supplier in a humidified incubator at 37° C. with 5% CO₂. For assays, 2500 cells/well were seeded in a 96 multi well plate in Eagle's Minimum Essential Medium (Sigma, M4655) with 10% fetal bovine serum (FBS) as recommended by the supplier.

Oligonucleotide Potency and Efficacy

Cells were incubated for 24 hours before addition of oligonucleotides. The oligonucleotides were dissolved in PBS and added to the cells at final concentrations of oligonucleotides was of 0.01, 0.031, 0.1, 0.31, 1, 3.21, 10, and 32.1 μM, the final culture volume was 100 μl/well. The cells were harvested 3 days after addition of oligonucleotide compounds and total RNA was extracted using the PureLink Pro 96 RNA Purification kit (Thermo Fisher Scientific), according to the manufacturer's instructions. Target transcript levels were quantified using FAM labeled TaqMan assays from Thermo Fisher Scientific in a multiplex reaction with a VIC labelled GAPDH control probe in a technical duplex and biological triplex set up. TaqMan primer assays for the target transcript of interest FNDC3B (Hs00981550_m1) and a house keeping gene GAPDH (4326317E VIC®/MGB probe). EC50 and efficacy of the oligonucleotides are shown in table 5 as % of control sample.

EC50 calculations were performed in GraphPad Prism6. The maximum FNDC3B knock down level is shown in table 5 as % of control.

TABLE 5
EC50 and maximal knock down (Max Kd) % of control

| CMP ID | | | Max | | |
|---|---|---|---|---|---|
| NO | EC50 | Std | kd | std | Start position(s) on SEQ ID NO: 1 |
| 11_1 | 3.33 | 0.74 | 44.50 | 4.67 | 54366, 54407, 54448, 54489, |
| | | | | | 54530, 54571, 54612 |
| 12_1 | 1.28 | 0.33 | 45.95 | 4.35 | 54366, 54407, 54448, 54489, |
| | | | | | 54530, 54571, 54612 |
| 13_1 | 0.52 | 0.03 | 8.95 | 1.10 | 54367, 54408, 54449, 54490, |
| | | | | | 54531, 54572, 54613 |
| 14_1 | 0.40 | 0.04 | 0.00 | 2.03 | 54368, 54409, 54450, 54491, |
| | | | | | 54532, 54573, 54614 |
| 15_1 | 95.92 | 473.37 | 0.00 | 98.74 | 351016 |
| 16_1 | 10.77 | 22.78 | 0.00 | 42.83 | 351016 |
| 17_1 | 10.12 | 8.99 | 78.71 | 8.22 | 351016 |
| 18_1 | 1.13 | 0.08 | 41.12 | 1.15 | 351018 |

1. An antisense oligonucleotide of 10 to 50 nucleotides in length,
which comprises a contiguous nucleotide sequence of 10 to 30 nucleotides in length which is at least 90% complementary, including fully complementary, to a mammalian FNDC3B encoding target nucleic acid, wherein the antisense oligonucleotide is capable of reducing the expression of the mammalian FNDC3B encoding target nucleic acid in a cell.

2. The antisense oligonucleotide according to claim 1, wherein the contiguous nucleotide sequence is at least 90% complementary, such as fully complementary to a sequence selected from the group consisting of SEQ ID NO: 1, 2, 3, 4, 5, 6, 7 and 8, or a naturally occurring variant thereof.

3. The antisense oligonucleotide according to claim 1, wherein the contiguous nucleotide sequence is fully complementary to the mammalian FNDC3B encoding target nucleic acid.

4. The antisense oligonucleotide according to claim 1, wherein the contiguous nucleotide sequence is at least 90% complementary, such as fully complementary, to an intron region present in the pre-mRNA of mammalian FNDC3B encoding target nucleic acid (e.g. SEQ ID NO: 1).

5. The antisense oligonucleotide according to claim 1, wherein the contiguous nucleotide sequence is at least 90% complementary, such as fully complementary, to an intron region present in the pre-mRNA of human FNDC3B, selected from position 145-72824 on SEQ ID NO: 1, position 72964-93844 on SEQ ID NO: 1, and position 93920-147084 on SEQ ID NO: 1.

6. The antisense oligonucleotide according to claim 1, wherein the contiguous nucleotide sequence is at least 90% complementary, such as fully complementary, to nucleotides 145-72824 of the human pre-mRNA of mammalian FNDC3B encoding target nucleic acid of SEQ ID NO: 1.

7. The antisense oligonucleotide according to claim 1, wherein the contiguous nucleotide sequence is at least 90% complementary, such as fully complementary, to SEQ ID NO: 9, 10, 19 or 20.

8. The antisense oligonucleotide according to claim 1, wherein the contiguous nucleotide sequence is at least 90% complementary, such as fully complementary, to a target region of SEQ ID NO 1, selected from the group consisting of position 54366-54385, 54407-54426, 54448-54467, 54489-54508, 54530-54549, 54571-54590, 54612-54631; 54367-54384, 54408-54425, 54449-54466, 54490-54507, 54531-54548, 54572-54589, 54613-54630; 54368-54383, 54409-54424, 54450-54465, 54491-54506, 54532-54547, 54573-54588, 54614-54629; 54366-54379, 54407-54420, 54448-54461, 54489-54502, 54530-54543, 54571-54584, 54612-54625; 351016-351033; 351016-351029; 351016-351035; and 351018-35133 of SEQ ID NO: 1.

9. The antisense oligonucleotide according to claim 1, wherein the contiguous nucleotide sequence is at least 90% complementary, such as fully complementary, to a target region of 10-22, such as 14-20, nucleotides in length of the target nucleic acid of SEQ ID NO: 1, wherein the target region is repeated at least 2 times across the target nucleic acid.

10. The antisense oligonucleotide according to claim 1, wherein the contiguous nucleotide sequence is at least 90% identical, such as is 100% identical to a sequence selected from the group consisting of SEQ ID NO 11, 12, 13, 14, 15, 16, 17 and 18.

11. The antisense oligonucleotide according to claim 1, wherein the contiguous nucleotide sequence consists of a sequence selected from the group consisting of SEQ ID NO 11, 12, 13, 14, 15, 16, 17 and 18.

12. The antisense oligonucleotide of claim 1, wherein the contiguous nucleotide sequence comprises one or more 2′ sugar modified nucleosides.

13. The antisense oligonucleotide of claim 12, wherein the one or more 2′ sugar modified nucleoside is independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, arabino nucleic acid (ANA), 2′-fluoro-ANA and LNA nucleosides.

14. The antisense oligonucleotide of claim 12, wherein the one or more modified nucleoside is a LNA nucleoside.

15. The antisense oligonucleotide of claim 1, wherein the contiguous nucleotide sequence comprises at least one modified internucleoside linkage.

16. The antisense oligonucleotide of claim 1, wherein at least 50%, such as at least 75%, such as at least 90%, such as all of the internucleoside linkages within the contiguous nucleotide sequence are phosphorothioate internucleoside linkages.

17. The antisense oligonucleotide of claim 1, wherein the antisense oligonucleotide is capable of recruiting RNase H.

18. The antisense oligonucleotide of claim 1, wherein the antisense oligonucleotide, or contiguous nucleotide sequence thereof, consists or comprises of a gapmer of formula 5′-F-G-F′-3′, where region F and F′ independently comprise 1-8 nucleosides, of which 1-5 are 2′ sugar modified nucleosides and defines the 5′ and 3′ end of the F and F′ region, and G is a region between 6 and 17 nucleosides which are capable of recruiting RNaseH, such as a region comprising 6-17 DNA nucleosides.

19. The antisense oligonucleotide of claim 1, wherein the antisense oligonucleotide, or contiguous nucleotide sequence thereof is selected from the group consisting of GaatttgctagtggtggtGG (Compound ID 11_1); GCTagtggtggtGG (Compound ID 12_1); AATttgctagtggtgGTG (Compound ID 13_1); ATTTgctagtggtgGT (Compound ID 14_1); TTatttctacagtttccAGA (Compound ID 15_1); ATTTctacagtttccAGA (Compound ID 16_1); CTacagtttcCAGA (Compound ID 17_1); and ATTTctacagtttCCA (Compound ID 18_1); wherein capital letters represent LNA nucleosides, such as beta-D-oxy LNA nucleosides, lower case letters represent DNA nucleosides, optionally, all LNA C are 5-methyl cytosine, and all internucleoside linkages are phosphorothioate internucleoside linkages.

20. A conjugate comprising the antisense oligonucleotide according to claim 1, and at least one conjugate moiety covalently attached to said oligonucleotide.

21. A pharmaceutically acceptable salt of the antisense oligonucleotide according to claim 1.

22. A pharmaceutical composition comprising the antisense oligonucleotide of claim 1, and a pharmaceutically acceptable diluent, solvent, carrier, salt and/or adjuvant.

23. An in vivo or in vitro method for inhibiting a mammalian FNDC3B expression in a target cell which is expressing the mammalian FNDC3B, said method comprising administering an antisense oligonucleotide of claim 1 in an effective amount to said cell.

24. A method for treating or preventing a disease comprising administering a therapeutically or prophylactically effective amount of an oligonucleotide of claim 1 to a subject suffering from or susceptible to the disease.

25. The method of claim 24, wherein the disease is selected from the group consisting of cancer, such as acute myeloid leukemia, or hepatocellular carcinoma.

26. The antisense oligonucleotide of claim 1 for use in medicine.

27. The antisense oligonucleotide of claim 1 for use in the treatment or prevention of cancer, such as hepatocellular carcinoma or acute myeloid leukemia.

28. Use of the antisense oligonucleotide of claim 1, for the preparation of a medicament for treatment or prevention of cancer, such as hepatocellular carcinoma or acute myeloid leukemia.
